(19) United States
(12) Patent Application Publication
(10) Pub. No.: US 2022/0112469 A1
(43) Pub. Date: Apr. 14, 2022

(54) Targeted Phage for Bacterial Detection and Destruction

(71) Applicants: Boris PETERLIN, San Francisco, CA (US); The Regents of the University of California, Oakland, CA (US)

(72) Inventors: Irene Chen, Los Angeles, CA (US); Huan Peng, Los Angeles, CA (US); Samuel Verbanic, Los Angeles, CA (US)

(73) Assignee: The Regents of the University of California, Oakland, CA (US)

(21) Appl. No.: 17/280,506

(22) PCT Filed: Oct. 2, 2019
(86) PCT No.: PCT/US2019/054361

§ 371 Date: Mar. 26, 2021

Related U.S. Application Data
(60) Provisional application No. 62/740,213, filed on Oct. 2, 2018.

(51) Int. Cl. C12N 7/00; A61K 45/06; A61K 35/76; A61K 47/69; G01N 33/569; G01N 33/543; G01N 33/553

(52) U.S. Cl. CPC C12N 7/00; A61K 45/06; A61K 35/76; B82Y 5/00; G01N 33/56911; G01N 33/54346; G01N 33/553; A61K 47/6929

(57) ABSTRACT

Novel chimeric proteins may be used to inhibit transcriptional A activities that are mediated by transcription factor interactions with P-TEFb. The chimeras contain elements that recruit the target transcription factor, maintain CDK9 in an inactive state, and competitively inhibit P-TEFb binding to the transcription factor. The chimeras may be configured for inhibition of HIV Tat mediated transcription and thus provide a novel means of preventing reactivation of integrated HIV, providing a new tool for emerging “block and lock” HIV cure strategies.

### CROSS-REFERENCE TO RELATED APPLICATIONS: This application is a 35

USC § 371 national stage application of International Patent Application Number PCT/US2019/054361, filed Oct. 2, 2019, which claims the benefit of priority to U.S. Provisional Patent Application Ser. No. 62/740,213 entitled “Phage Targeted Gold Nanoparticles for Detection and Cell Killing in Bacterial Infections,” filed Oct. 2, 2018, the contents of which applications are hereby incorporated by reference.

### STATEMENT REGARDING FEDERALLY SPONSORED RESEARCH OR DEVELOPMENT

This invention was made with government support under grant number 1DP2GM123457-01 awarded by the National Institutes of Health and grant number W911NF-09-D-0001 awarded by the US Army Research Office. The government has certain rights in the invention.

### BACKGROUND OF THE INVENTION

Antibiotic-resistant bacterial infections, particularly from gram-negative organisms, are widely recognized as an urgent threat to health worldwide. Accordingly, there is a need in the art for new agents and methods for killing antibiotic-resistant bacteria.

Similarly, conventional bacterial detection methods, including culturing, ELISA, and polymerase chain reaction (PCR) methods, have inherent and significant drawbacks, such as long processing times and the need for specialized reagents and equipment. Accordingly, there is also a need in the art for new platforms for the rapid and facile detection of specific bacterial species. A versatile and effective detection platform would have many potential applications, for example, in medicine, environmental applications, and food safety.

Bacteriophages or often called phages, are abundant and ubiquitous, and represent highly evolved and very efficient systems of bacterial targeting. Phages have evolved multiple mechanisms to target their bacterial hosts, such as high-affinity, environmentally hardy receptor-binding proteins. The unique selective and efficient targeting abilities of phages suggest that they could be applied to solve various problems in the area of bacterial detection and treatment of infection.

For example, the use of phage cocktails to treat bacterial infection by a diverse collection of phage has been explored, for example, as described in Chan and Abeton, Phage Therapy Pharmacology: Phage Cocktails, 2012, Advances in Applied Microbiology, Ch 1, 1-23. Drug-conjugated phage as a delivery platform for treating infection has been demonstrated as well, for example in Yacoby et al., Targeted Drug-Carrying Bacteriophages as Antibacterial Nanomedicines, ANTIMICROBIAL AGENTS AND CHEMOTHERAPY, June 2007, p. 2156-2163. The use of phage to detect bacteria has been tested by various groups, for example, as described in Klumpp and Loessner, Detection of Bacteria with Bioluminescent Reporter Bacteriophage. Adv. Biochem. Eng./Bio-technol. 2014, 144, 155-171 and van der Merwe, et al., Phage-Based Detection of Bacterial Pathogens. Analyst 2014, 139, 2617-2626.

However, despite these previous efforts, phages have not achieved mainstream or widespread use in therapeutic, clinical, and analytical techniques. This is likely due to the unique challenges posed by this complicated organism. Phages are replicating, evolvable entities whose biology is poorly understood. There is a severe lack of biological characterization for most phage types, which may carry toxin genes or cause generalized transduction of bacterial genes. In addition, the pharmacokinetics and pharmacodynamics of phages are difficult to model due to their exponential replication and rapid evolution, presenting a major barrier to clinical translation. Exponential replication may also lead to undesirably rapid release of bacterial endotoxins, harming patients. Accordingly, substantial barriers exist that have prevented the widespread use of phage in the control and detection of bacteria.

### SUMMARY OF THE INVENTION

The scope of the invention encompasses novel functionalized phages which may be used in diverse applications such as bacterial detection and the control of bacterial infections. The scope of the invention encompasses novel bacteriophage compositions that combine customized host specificity with powerful plasmonic properties.

In a first aspect, the scope of the invention encompasses modification of well characterized phage types in order to enable their targeting to selected bacterial types, such as pathogenic bacteria, for example, antibiotic resistant bacteria. In some implementations, the functionalized phages of the invention are modified to express receptor binding proteins that confer host specificity to a selected target bacteria.

In another aspect, the phages of the invention are functionalized with nanoparticles, particularly plasmonic nanoparticles, wherein the aggregation of these functionalized phages results in useful plasmonic resonance emissions that can be exploited for medical, research, and other uses.

In some implementations, the functionalized phages of the invention are functionalized with gold, silver, or other metallic nanoparticles having high plasmon resonance when excited by suitable light or other energy. In some implementations, the metallic nanoparticles are nanorods, with highly tunable plasmonic responses. In some embodiments, the nanorods are gold nanorods that are excitable by near infrared wavelengths.

In some embodiments, excitation of the nanoparticles of the functionalized phages results in plasmonic resonance-mediated localized thermal effects. In one aspect, the scope of the invention is directed to the killing of target bacteria by application of functionalized phages of the invention, wherein such phages selectively aggregate on target bacterial cells and wherein excitation of phage-mediated aggregates of nanoparticles creates non-radiative heating that kills the targeted bacterial cells while sparing the surrounding host cells and non-target bacterial types. This therapeutic method also destroys the phage, obviating potential complications from the applied phage propagating and evolving.

In some embodiments, excitation of the functionalized phages results in plasmonic responses that strongly affect optical emissions. In one aspect, the scope of the invention is directed to the detection of target bacteria by application of functionalized phages of the invention, wherein such phages selectively aggregate on target bacterial cells. Measurement of optical signals that are sensitive to the abundance of the aggregated nanoparticles enables the detection and quantification of the targeted bacterial cells.

The various functionalized phages of the invention and methods of using them disclosed herein advantageously provide the art with novel therapeutic, clinical, research, analytical, and industrial tools for the treatment of bacterial infections, bacterial control in other contexts, and for detection and analytical methods.

### BRIEF DESCRIPTION OF THE DRAWINGS

FIGS. 1A, 1B, 1C, and 1D. 1A: Schematic of the steps in making phage-AuNR bioconjugates for bacterial detection and cell-killing. Phage (101) with wild type RBP (102) is engineered to instead express the RBP (103) from another phage which is directed to target bacteria. Chemical modification (SATP) introduced thiol groups (105) along the phage coat, followed by conjugation with gold nanorods (106), resulting in a re-targeted phage functionalized with gold nanorods (107). FIG. 1B: Phage-AuNR bioconjugates are introduced to a target region of mammalian cells 110, containing both non-target bacteria 109 and target bacteria 108. FIG. 1C: The functionalized phage 107 selectively aggregate on the target bacteria. FIG. 1D: Upon exposure to light, localized heating from the aggregated nanorods destroy the target bacteria.

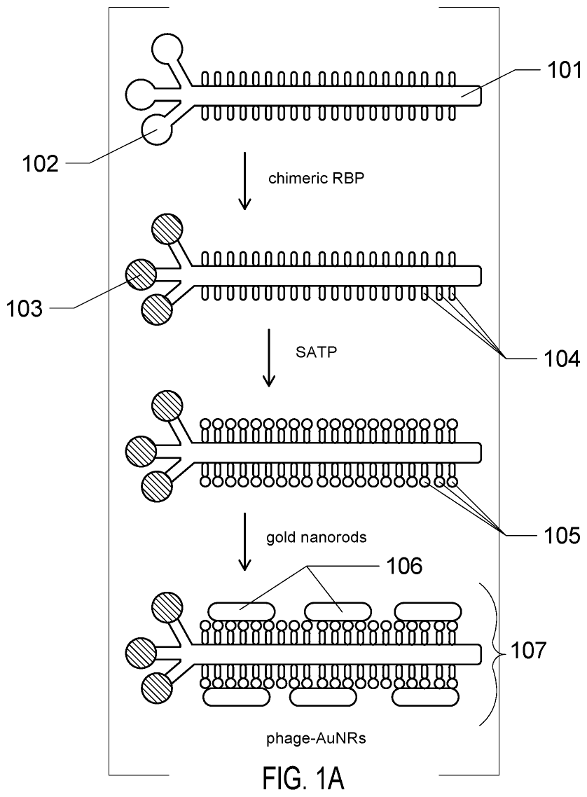

FIG. 2. FIG. 2 depicts the UV-vis spectrum of AuNR alone, M13KE-AuNR, and M13KE-AuNR in the presence of E. coli cells at 10², 10⁴ and 10⁶ CFU.

FIGS. 3A and 3B. Detection of P. aeruginosa. FIG. 3A: UV-vis spectra of AuNR, phage-AuNR, and phage-AuNR with P. aeruginosa at 10², 10⁴, and 10⁶ CFU. FIG. 3B. Sensitivity of P. aeruginosa detection in the context of a mixture of bacteria (E. coli (F⁻), V. cholera, X. campestris (pv vesicatoria), X. campestris (pv campestris) and E. coli (I⁻)). The target cells P. aeruginosa were present in the amount indicated in the legend; the other bacterial species were present at 10⁶ CFU each. The spectra of AuNRs and M13-g3p(Pf1)-AuNR bioconjugates are also shown.

FIG. 4. FIG. 4 depicts loss of colony-forming units at different irradiation time points (normalized to untreated control) for target and non-target E. coli plated on LB plates. Error bars show one standard deviation calculated from three or more replicates.

FIG. 5. FIG. 5 depicts the viability of biofilm and MDCKII cells treated with M13-g3p(Pf1)-AuNRs by PrestoBlue cell viability assay for M13-g3p(Pf1)-AuNR treatment of MDCKII cells grown alone, P. aeruginosa biofilm grown on MDCKII cells, and P. aeruginosa biofilm grown alone during the photothermal cell lysis experiment at different irradiation time points. In this assay, the PrestoBlue reagent is modified by the reducing environment of live cells and fluoresces; both MDCKII and P. aeruginosa cells contribute to PrestoBlue fluorescence. MDCKII cells are largely viable while P. aeruginosa cells are killed over the irradiation time course. As expected, the fluorescence of the biofilm grown on MDCK cells is roughly equal to the sum of the fluorescence of MDCKII cells alone plus the fluorescence of biofilm cells alone. After 6 minutes, the fluorescence of the biofilm grown on MDCKII cells appears to be largely attributable to MDCKII cells alone, consistent with selective killing of P. aeruginosa.

FIGS. 6A and 6B. FIG. 6A: Heating profiles of AuNRs (3.3 nM AuNRs), M13KE-AuNR (3.3 nM AuNRs, 10¹¹ phage/mL), M13KE-AuNR mixed with ER2738 (10⁶ cells/mL), and water (control) upon irradiation with the 808 nm laser for 10 min. AuNR concentrations were measured by single particle ICP-MS. FIG. 6B: The overlap of LSPR spectra of the AuNR bioconjugates with the laser is shown.

FIGS. 7A and 7B. FIG. 7A: For E. coli ER2738 cells incubated with M13KE-AuNR bioconjugates after photothermal lysis for 10 min, fluorescence spectra of BCECF at different temperatures was used to create a calibration curve. FIG. 7B: Local (cell) temperature and bulk temperature in the solution upon irradiation in presence of M13KE-AuNRs, measured by BCECF fluorescence.

FIG. 8. Schematic for the detection of target bacteria. Phage expressing wild type RBP is engineered to express a foreign RBP. This is folllwed by thiolation of coat proteins by EDC chemistry. The thiolated chimeric phages are added to media containing bacteria (rounded rectangle) and may attach to the cells. Centrifugation separates cell-phage complexes from free phage. The pellet is resuspended in solution with gold nanoparticles (white circle), whose aggregation on the thiolated phage produces a color change.

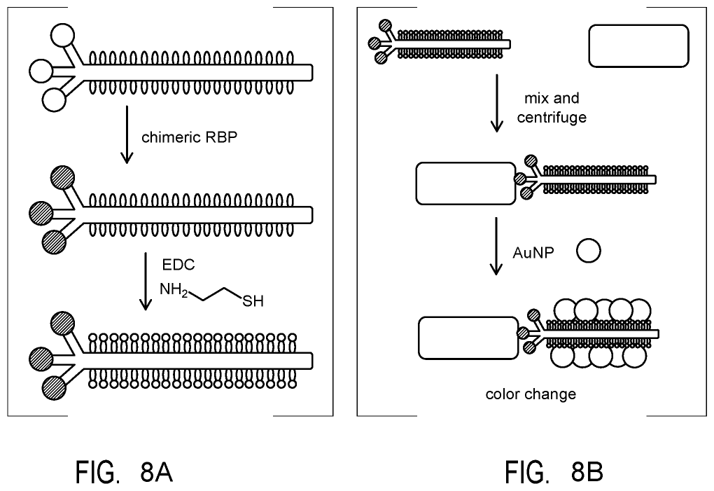

FIGS. 9A, 9B, and 9C. FIGS. 9A, 9B, and 9C depict UV-vis spectra for detection of target bacteria in different medium. For each panel, samples contain AuNPs alone, control unmodified phage with 10⁶ CFU host bacteria, and thiolated phage with host bacteria at 10², 10⁴, and 10⁶ CFU FIG. 9A: V. cholerae 0395 in seawater. FIG. 9B: P. aeruginosa in tap water. FIG. 9C: E. coli (I⁺) in tap water.

### DETAILED DESCRIPTION OF THE INVENTION

The scope of the invention encompasses novel compositions of matter comprising functionalized phages. A first feature of the functionalized phage of the invention is that it may be engineered to have specificity and affinity for a selected type of target bacteria. This is achieved by the introduction of receptor binding proteins derived from other phages. A second feature of the functionalized phage of the invention is that it is decorated with a plurality of plasmonic nanoparticles, such as gold nanorods. When the functionalized phages of the invention encounter their target bacteria, they adsorb with high affinity, creating aggregations of the plasmonic nanoparticles. When light of the right properties is applied to such target-induced aggregations, intense plasmonic effects are generated, such as highly localized non-radiative heating and altered optical emissions. These effects may be exploited for applications such as bacterial cell killing and bacterial detection and quantification, as disclosed herein. The various elements of the invention are described next.

Target Bacteria. The novel phages of the invention and associated methods of using such phages encompass the selective binding of phage to a target bacteria type. As used herein, “target bacteria” will refer to cells of one or more types of bacteria to which the phage selectively and effectively adsorbs. Target bacteria may comprise bacterial genera, species, subtypes, serovars etc. which to which a phage type will preferentially adsorb or associate with. Exemplary target bacteria include bacteria of the genera Escherichia, Shigella, Salmonella, Enterobacter, Yersinia, Vibrio, Legionella, Pseudomonas, Neisseria, Bordetella, Helicobacter, Listeria, Staphylococcus, Streptococcus, Enterococcus, Clostridium, Corynebacterium, Mycobacterium, Treponema, Borrelia, Campylobacter, Chlamydia, Haemophilus, Serratia and Klebsiella. In various embodiments, the target bacteria may comprise an antibiotic resistant strain of bacteria, for example, Acinetobacter baumannii, for example, carbapenem-resistant types; Pseudomonas aeruginosa, for example, carbapenem-resistant types; Enterobacteriaceae, for example, carbapenem-resistant, ESBL-producing types; Enterococcus faecium, for example, vancomycin-resistant types; Staphylococcus aureus, for example, methicillin-resistant and vancomycin-resistant types; Helicobacter pylori, for example, clarithromycin-resistant types; Campylobacter spp., for example, fluoroquinolone-resistant types; Salmonellae, for example, fluoroquinolone-resistant types; Neisseria gonorrhoeae, for example, cephalosporin-resistant and fluoroquinolone-resistant types; Streptococcus pneumoniae, for example, penicillin-non-susceptible types; Haemophilus influenzae, for example, ampicillin-resistant types; and Shigella spp., for example, fluoroquinolone-resistant types.

The compositions and associated methods of the invention are especially amenable to the detection or destruction of bacterial cells, and the description herein will make reference to “target bacteria” as the target cells. However, it will be understood that the target cells may be of any kind, including bacterial cells, eukaryotic microbes such as yeast, and or other cell types that can be targeted by phage.

Phages. The methods and compositions of the invention encompass functionalized phages. As used herein, “phage” will refer to bacteriophages, as known in the art, encompassing any bacteriophage or other viral organism or construct capable of infecting bacterial cells. The phage may be of any type, serotype, or species. Exemplary phages include phages of Myoviridae, Siphoviridae, Podoviridae, Tectiviridae, Corticoviridae, Lipothrixviridae, Plasmaviridae, Rudiviridae, Fuselloviridae, Inoviridae, Microviridae, Leviviridae, Cystoviridae, Ampullaviridae, Bicaudaviridae, Clavaviridae, Globuloviridae or Guttavirus.

The phage type may be selected based on its selectivity for specific host types, i.e. its affinity for adsorption or binding to a selected target bacteria. In some implementations, the phage is a natural, non-genetically modified phage, having a native or evolved affinity or specificity for a selected target bacteria. Generally, however, for use in therapeutic and analytical platforms, it will be advantageous to use well characterized phages that have been genetically modified to alter host selectivity. Likewise, it will generally be preferred to use a phage type that is readily propagated and/or engineered by established protocols. Exemplary phages that are well characterized, readily propagated, and easily genetically modified include, for example: M13, MS2, T4, T5, T7, K1F, K11, fd, f1 or SP6 phages.

The phage of the invention will comprise phages expressing one or more targeting moieties. The targeting moiety will comprise any peptide or other composition of matter that facilitates the adsorption or binding of the phage to a target bacteria. Such adsorption is facilitated by the interactions of the targeting moiety with complementary moieties present on the surface of the target bacteria. For example, complementary moieties may include polysaccharides, lipopolysaccharides, carbohydrates, extracellular protein domains, flagella, pili, teichoic acids, and other moieties by which adsorption or binding of phage may be facilitated.

In a primary embodiment, the targeting moiety will comprise a receptor binding protein (RBP). RBP's are typically located in phage tail fibers, spikes, or baseplates and they facilitate the initial, specific interaction of the phage with their target bacteria. Numerous RBPs have evolved, which may confer high affinity and specificity for specific classes, species, subtypes, or serovars of bacterial cells.

In one embodiment, the phage of the invention is a phage that naturally, without genetic modification, express one or more RBPs that confer specificity for a selected target species. In a primary implementation, the phage of the invention is genetically modified to express one or more heterologous RBPs, i.e., RBPs from different phage types, imparting a new host range to the phage.

RBPs are typically elements of phage structures, including: phage tail fibers, including short side tail fibers or long tail fibers; tail spikes; tail shafts; short tail tip fibers; minor coat proteins; or protruding baseplate proteins. In one embodiment, the RBP is a minor coat protein or protein domain which is presented on the surface of the phage.

The scope of the invention encompasses phage comprising any selected RBP, including known RBPs, novel RBPs isolated from natural phage populations, or synthetic RBPs or created by artificial selection or recombinant technologies. Exemplary RBPs include T4 gp37, gp38, T7 gp17, T3 gp17, P22 gp9, SP6 gp46, K1-5 gp46 K1-5 gp47, K1F gp17, K1E gp47, K11 gp17, phiSG-JL2 gp17, phiMB-PF7A gp17, 13a gp17, Pf1 g3p analog and CTXphi g3p analog, 77 ORF104 (which targets Staphylococcus aureus, ad described in Viruses 2019, 11, 268; doi:10.3390/v11030268) and other RBPs known in the art.

Phages may be genetically modified to express heterologous RBPs (or portions thereof, wherein such portions are sufficient to facilitate phage adsorption to target bacteria). The genetic modification may be achieved by any number of methods known in the art. In one embodiment, the native RBP genes of the genetically modified phage may be swapped or replaced with homologous sequences coding for a different RBP that confers a different host specificity. In various embodiments, the phage is engineered by replacing the host-binding elements of native tail fibers, tail spikes, short tail tip fibers, or baseplate proteins with heterologous elements that bind different targets.

Genetic modification of phages may be achieved by means known in the art for phage engineering, including:
homologous recombination methods (for example, as described in Mahichi et al., Site-specific recombination of T2 phage using IP008 long tail fiber genes provides a targeted method for expanding host range while retaining lytic activity, FEMS Microbiol Lett. 2009 June; 295(2):211-7);
bacteriophage recombineering of electroporated DNA (BRED) (for example, as described in Marinelli et al., Recombineering: A powerful tool for modification of bacteriophage genomes, Bacteriophage. 2012 Jan. 1; 2(1):5-14 and Marinelli et al., 2008. BRED: a simple and powerful tool for constructing mutant and recombinant bacteriophage genomes. PLoS One 3:e3957. doi:10.1371/journal.pone.0003957);
in vivo recombineering (for example, as described in Oppenheim et al., In vivo recombineering of bacteriophage lambda by PCR fragments and single-strand oligonucleotides, Virology. 2004 Feb. 20; 319(2):185-9);
CRISPR-Cas mediated genome engineering (for example, as described in Kiro et al., Efficient engineering of a bacteriophage genome using the type I-E CRISPR-Cas system, RNA Biol. 2014; 11(1):42-4 and Martel et al., CRISPR-Cas: an efficient tool for genome engineering of virulent bacteriophages, Nucleic Acids Res. 2014 August; 42(14):9504-13;
in vitro refactoring (for example, as described in Chan et al., Refactoring bacteriophage T7, Mol Syst Biol. 2005; 1:2005.0018;)
yeast based assembly of phage genomes (for example, as described in Jaschke et al., A fully decompressed synthetic bacteriophage øX174 genome assembled and archived in yeast, Virology. 2012 Dec. 20; 434(2):278-84); and
phage display technology platforms maybe create fusion proteins with phage coat proteins, wherein introduced or novel sequences are displayed on the phage coat, which such sequences can be used to bind target bacteria and which can be evolved to discover new target binding moieties.

In one implementation, the genetic modification of phage is achieved by the manipulation and transformation of isolated phage genomes. For example, a phage genome may be modified to introduce restriction sites that flank native RBP sequences (or domains thereof). The native sequences can be removed by restriction digest and replaced with sequences coding for one or more selected RBPs. For example, as described in the Examples herein, an M13 phage genome comprising introduced restriction sites flanking the sequence coding for the N-terminal domain of the g3p RBP may be utilized. Engineered DNA sequences coding for a selected replacement RBP may be ligated into the M13 genome. Following genetic manipulation, the modified phage genome is transformed into host bacterial cells to propagate the engineered phage expressing the new RBP.

It will be understood that functional equivalents of RBPs, proteins capable of facilitating selective binding to target bacteria, may be utilized in place of or in combination with phage-derived RBPs. The targeting moiety may comprise any peptide, protein, or composition of matter that facilitates phage adsorption, binding, or other selective association with the target bacteria. For example, in one embodiment, the targeting protein is a protein or polypeptide with specificity for eukaryotic microbes such as yeast. In one embodiment, the targeting polypeptide is a receptor having a complementary ligand on a target cell surface, such as an extracellular protein domain, a carbohydrate moiety, or a bacterial lipid. In some embodiments, the targeting polypeptide is a sequence derived from the antigen-binding region of an antibody having high affinity for target cell epitopes. In some implementations, the RBP is an engineered sequence comprising a hybrid, synthetic, or otherwise non-natural RBP sequence.

In an alternative implementation, the phage may be engineered to express an affinity tag or other conjugation moiety, for example, being expressed at the terminal ends of tail fibers, tail spikes, or baseplate proteins. Exemplary affinity tags include, for example one member of a SpyCatcher-SpyTag system, SnoopCatcher-SnoopTag system, DogTag tagging system; Isopeptag tagging system; SdyTag tagging system; biotin-avidin tagging systems; strepavidin-biotin tagging systems; or polyhistidine tagging systems, as known in the art. Such phages may be functionalized with RBPs or other target-binding moieties bearing complementary tags.

Plasmonic nanoparticles. The phages of the invention comprise phages functionalized with nanoparticles which impart useful properties to the phage. In a primary implementation, the nanoparticles are plasmonic nanoparticles. Plasmonic nanoparticles, as referred to herein, are particles having certain electron density characteristics that render them excitable when exposed to light (or other electromagnetic energy) at specific frequencies. When excited, electronic oscillation occurs and the resulting energy is dissipated in ways that impart interesting properties to the nanoparticles. Aggregations of excited nanoparticles can create highly localized and intense thermal and optical emissions that may be harnessed for various applications, as set forth herein.

The plasmonic properties of nanoparticles are determined by the composition of the nanoparticle, the size of the nanoparticle, and the shape of the nanoparticle. Regarding the composition of the nanoparticles, any composition of matter having a resonant plasmonic response to energetic exposure may be used. In a primary implementation of the invention, plasmonic nanoparticles will be metals having sufficient free electrons to induce desired plasmon behaviors. In one embodiment, the plasmonic nanoparticles of the invention comprise gold, for example, pure gold. In one embodiment, the plasmonic nanoparticles of the invention comprise silver, for example, pure silver. The plasmonic nanoparticles of the invention comprise may comprise a metal selected from the group consisting of copper, aluminum, iron, iron oxides, zinc, cadmium, lanthanum, lead, tin, mercury, other metals, or alloys of the foregoing. In some embodiments, the nanoparticle is a semiconductor material, for example, an organic or organoometallic composition. In one embodiment, the nanoparticle comprises a carbon nanotube or graphene composition. Metals such as gold, silver, copper, and aluminum advantageously exhibit plasmon resonance when excited by light in the near-infrared and visible wavelengths.

Plasmonic properties are particularly affected by the shape of the nanoparticles. Resonant oscillations of the excited nanoparticles are determined by localized charge accumulations, which are dictated by the shape of the particles. The plasmonic nanoparticles of the invention may include nanorods, nanofiliaments, nanospheres, nanostars (for example, a core structure having multiple branches or projections, for example, as described in Khan et al., Facile synthesis of gold nanostars over a wide size range and their excellent surface enhanced Raman scattering and fluorescence quenching properties, Journal of Vacuum Science & Technology B 36, 03E101 (2018), or Pallavicini et al., 2015, Gold Nanostar Synthesis and Functionalization with Organic Molecules, n: Gold Nanostars. SpringerBriefs in Materials. Springer, Cham), nanotubes or other geometries such as triangular prisms, nanocubes, or nanocages.

The size of the nanosphere, nanorod, nanostar, nanofiliament, or other shape may vary. Exemplary sizes are in the range of 0.01-100 nm, for example, in the range of 2-20 nm. For example, the nanoparticle may comprise a particle having a maximual length, width, diameter, etc. of about (i.e., within 5%, 10%, or 20% of) 0.5 nm, 1 nm, 2 nm, 3 nm, 4 nm, 5 nm, 6 nm, 7, nm, 8 nm, 9, nm, 10 nm, 11 nm, 12 nm, 13 nm, 14 nm, 15 nm, 16 nm, 17, nm, 18 nm, 19, nm, 20 nm, 21 nm, 22 nm, 23 nm, 24 nm, 25 nm, 26 nm, 27, nm, 28 nm, 29, nm, or 30 nm.

In a primary embodiment, the plasmonic nanoparticles of the invention comprise nanorods. Advantageously, nanorods have substantial plasmonic resonance properties, which may be tuned by selecting the aspect ratio of the nanorod. In one embodiment, the plasmonic nanoparticles of the invention comprise nanorods, for example, gold or silver nanorods. In one embodiment, the nanorod is a nanorod with a length in the range of 1-50 nm, for example 2-25 nm, for example 4-8 nm. In one embodiment, the nanorod may have width in the range of 1-20 nm, for example, between 1-5 nm. In one embodiment, the aspect ratio (length:width) of the nanorod is about 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, or 20:1.

The composition, size, and shape, of the plasmonic nanoparticles of the invention may be selected based on the desired end use. In the case of imaging methods, as described herein, plasmonic nanoparticles having large light scattering or absorption effects are desired. In the case of bacterial cell killing applications, as described herein, desirable plasmonic nanoparticles are those capable of intense localized non-radiative thermal decay as a result of Light-induced oscillations. Nanoparticle materials and configurations can be designed based on the known properties of existing nanomaterials, or may be designed by application of plasmon modeling tools known in the art, for example, by the use of Maxwell equations, Gans theory, dipolar approximations, and other tools known in the art.

Other Nanoparticles. The scope of the invention further encompasses phage functionalized with non-plasmonic functional nanoparticles, for example, in place of or in addition to plasmonic nanoparticles. Non-plasmonic functional nanoparticles may comprise any functional moiety, for example: quantum dots (for imaging applications), magnetic nanoparticles (iron, iron oxides, and other magnetic, paramagnetic, or supermagentic materials); drug binding or drug-loaded nanoparticles (such as dendrimers, hydrogels, carbon nanotubes, liposomes, vesicles, caging molecules, and other drug delivery particles known in the art).

Phage Functionalization. The functionalization of phage with nanoparticles, for example, plasmonic nanoparticles, may be accomplished by any number of chemistries. Reactive moieties present on coat and/or capsid proteins may be used for conjugation of hundreds to thousands of nanoparticles per phage. For example, in one implementation, solvent-accessible carboxyl groups of glutamic or aspartic acid residues may serve as conjugation sites using coupling chemistries known in the art.

Other conjugation sites on phage coat or capsid proteins include solvent accessible free amines of lysine residues. For example, stable amide linkages to functional moieties may be formed at such sites utilizing N-hydroxysuccinimide (NHS) esters, isothiocyanates, isocyanates, or acyl azides, as known in the art.

In other implementations, functionalization is achieved by conjugation of functional moieties to solvent-accessible tyrosine residues. For example, conjugation to tyrosine may be achieved by the use of diazonium groups. Diazonium may additionally be utilized in the modification of lysine or histidine residues.

Other methods of phage modification are known in the art, for example, modification of solvent accessible N-terminal amines. For example, N-terminal transamination/oxime chemistries may be utilized to functionalize coat proteins. In one embodiment, functionalization is achieved by reacting accessible amines of phage coat proteins with NETS-modified nanoparticles, for example, silver nanoparticles.

In some implementations, the phage genome is genetically modified to produce coat and/or capsid proteins comprising added or substituted amino acids at selected sites to facilitate functionalization, for example, glutamic acid, aspartic acid, tyrosine, lysine, threonine, serine, or cysteine residues. For example, the thiol groups of solvent accessible cysteine residues in coat proteins may be used as reactive handles, including native cysteines, and introduce cysteine residues. In another embodiment, genetic modification of coding sequences of coat proteins is used to introduce codons for the introduction of unnatural amino acids by suitable expression systems, wherein the subsequently incorporated non-natural amino acids are used as reactive moieties to conjugate functional moieties to the phage. In another implementation, specific peptide sequences are introduced to the coat proteins that facilitate capture of nanoparticles, for example, as described in Wang et al., Ultrasensitive Rapid Detection of Human Serum Antibody Biomarkers by Biomarker-Capturing Viral Nanofibers, ACS Nano, 2015, 9, 4475-4483 and Zhou et al., Phage-mediated counting by the naked eye of miRNA molecules at attomolar concentrations in a Petri dish, Nature Materials 2015, 14, 1058-1064).

In a primary implementation, phages are thiolated to facilitate conjugation with metal nanoparticles, for example, gold nanoparticles. For example, thiolation of phage coat proteins may be achieved by reacting accessible carboxyl groups of phage coat proteins, for example at solvent accessible glutamic acid or aspartic acid residues, with aminothiol compositions. For example, coupling to aminothiol compositions, comprising any water-soluble molecules with amine group at one end and thiol group at the other end, such as cysteamine, 3-Amino-1-propanethiol hydrochloride, 3-Aminopropane-1-thiol hydrochloride hydrate may be achieved by bioconjugation reagents such as carbodiimides, for example, as 1-ethyl-3-(-3-dimethylaminopropyl) carbodiimide hydrochloride (EDC) or N′,N′-dicyclohexyl carbodiimide (DCC). In one embodiment, the phage is M13 phage. In one embodiment, the M13 g3p coat protein is thiolated by reaction with aminothiols and EDC or functionally equivalent reagents.

Thiolated phage, i.e. phage comprising coat and/or capsid proteins bearing thiol groups, may then be further reacted with one or more type of nanoparticles to produce the fully functionalized phages of the invention, i.e. phages bearing nanoparticles such as gold nanorods.

Phage may be maintained and propagated on bacterial cultures. For functionalization, phages may be isolated or purified by means known in the art, for example, by centrifugation, for example cesium or saccharose gradient centrifugation. Alternatively, chromatography, for example, affinity chromatography, may be utilized.

An intermediate treatment process comprising a reaction or series of reactions may be required to render the phage competent for conjugation with functional nanoparticles. In one embodiment, the treatment process comprises the thiolation of phage proteins, for example, thiolation of coat and/or capsid proteins.

The final conjugation of nanoparticles to the phage may be achieved by any method known in the art, for example, incubation with a solution of the selected nanoparticles under suitable conditions for conjugation of the nanoparticles, for example incubation of thiolated phage with gold, silver or other metallic nanoparticles under suitable conditions, for example, the reaction can be performed in 4° C. from several hours to overnight under stirring or rotation. The pH can be adjust from pH 3 to 10. In an alternative implementation, the intermediate treatment process is not necessary and the nanoparticles are conjugated directly to the phage in a single reaction. Following functionalization, a purification process may be applied to isolate functionalized phages from the reaction mixture, for example by centrifugation, affinity chromatography or other methods.

The resulting phages will comprise phages functionalized with a plurality of nanoparticles. Nanoparticle density will depend on the number of reactive conjugation sites on the phage and the efficiency of the functionalization process. Nanoparticle abundance of tens to thousands of nanoparticles per phage may be achieved, for example, between 5-10, 20-20, 20-30, 30-40, 40-50, etc. nanoparticles per phage. For example, in the Examples set forth below, functionalization with gold nanorods averages about fifteen nanoparticles per phage.

Functionalized phages of the invention may be stored for later use, for example, under refrigeration, cryopreservation, or lyophilization, for example, in suitable buffers, cryopreservation solutions, or other suitable carriers.

In one implementation, thiolated phage is produced but is not functionalized with plasmonic nanoparticles until after its exposure to the target bacteria. For example, in certain detection methods, described later herein, thiolated phage is introduced to target bacteria, resulting in adsorption and aggregation of the thiolated phage on the target cells. Next, nanoparticles are provided, under conditions suitable for conjugation, resulting in the functionalization of the phage. For example, thiolated phage may be adsorbed to target bacteria and then subsequently functionalized with gold nanoparticles, e.g. gold nanorods.

Pharmaceutical compositions. In the case of functionalized phages for bacterial cell killing applications, these may be formulated in a pharmaceutical composition. The pharmaceutical composition may comprise phage admixed in any number of pharmaceutically acceptable carriers, including buffers, excipients, preservatives, diluents, encapsulating materials, releasing agents, coating agents, antioxidants, and other materials known in the art. Pharmaceutical compositions will be formulated according to the contemplated delivery method, for example, for intravenous, intra-arterial, intraperitoneal, intrapulmonary, oral, inhalation, intravesicular, intramuscular, intra-tracheal, subcutaneous, transmucosal, and transdermal applications. For example, in one embodiment, the phages are encapsulated using biocompatible polymers like polyethylene glycol or polylactic acid to form hydrogel/microgels.

Thermotherapy and Bacterial Killing Applications. In one implementation, the functionalized phages of the invention are utilized in the selective killing of target bacteria. By these methods, the functionalized phages of the invention may be used in the treatment or prevention of bacterial infections, sterilization applications, or other contexts wherein one or more target bacteria are to be destroyed or otherwise inhibited, such as food safety applications, water purification, or environmental remediation.

Such applications may be referred to as thermotherapy applications. In the general thermotherapy method of the invention, selective bacterial killing is achieved by thermal ablation. Plasmonic nanoparticles such as gold nanorods or nanostars exhibit intense surface plasmon resonance upon excitation by with suitable wavelengths of light. This energy is released primarily as non-radiative heat, leading to highly localized and strong temperature increases, for example, heating of up to fifty degrees Celsius above surroundings. This form of energy has a very short half-length, for example, ranging from the submicron range for a single nanoparticle to a few microns for an ensemble of nanoparticles. Accordingly, the heating is intense, but highly localized around the nanoparticle aggregates, resulting in the death of adsorbed bacterial cells, but avoiding lethal heating of nearby native cells or other non-target cells. Advantageously, this treatment also destroys the phage as well as the target bacteria, preventing or reducing off-target replication of the phage.

In a general thermotherapy application of the invention, the process is as follows:
A method of selectively killing target bacteria in/on a subject, material, or target structure by the steps of
introducing to the subject, material, or target structure a plurality of a plasmonic nanoparticle-functionalized phages which selectively adsorb to a selected target bacteria, creating nanoparticle aggretates; and
applying an energetic treatment to the subject, material, or target structure, wherein the applied energy is of a wavelength and intensity sufficient to induce plasmonic resonant excitation of the plasmonic nanoparticles, wherein the excitation results in the release of energy in the form non-radiative localized heating that kills the adsorbed bacterial cells and destroys the functionalized phage.

In one embodiment the nanoparticle is a gold nanorod or gold nanostar.

In one implementation, the thermotherapy method of the invention is applied in a subject. The subject may be any organism, including, in one embodiment, an animal for example, an animal subject at risk of infection by the selected target bacteria or suffering from an infection by the selected target bacteria. In one embodiment, the subject is a human. In other embodiments, the subject may be a non-human animal, such as a test animal, veterinary subject, or farm animal. In one embodiment, the subject organism is a plant, such as a crop plant. In one embodiment, the functionalized phages of the invention are deployed to a target site, comprising, for example, a wound, abscess, lesion, an organ, a compartment of the body, or any other selected target region. In various implementations, the application of functionalized phages of the invention is achieved by topical, intravenous, intra-arterial, intraperitoneal, intrapulmonary, oral, inhalation, intravesicular, intramuscular, intra-tracheal, subcutaneous, transmucosal, or transdermal delivery. Such administration may be in the form of a pharmaceutical composition comprising the functionalized phages of the invention.

The functionalized phages of the invention will be administered in a pharmaceutically efficient amount, encompassing an amount sufficient to induce a measurable therapeutic, biological, or bacterial killing effect. In one embodiment, the dosage is expressed a plaque forming units (PFU), with exemplary dosages of 10³ to 10¹⁵ PFU per dosage, for example, 10¹⁰ to 10¹³ PFU per gram of treated tissue. In one embodiment, the dosage is expressed as number of phages administered, for example, dosages in the range of billions or more of phages, for example, one to ten billion phages per dosage, 10 to 50 billion phages per dosage, or 50-100 billion phages per dosage. In one embodiment, the dosage is expressed mass of administered phage, for example 500-1000 ng administered phage.

In alternative implementations, the phage of the invention is not applied to a living organism, but is applied to a target structure, material or surface, such as a medical instrument, surfaces in a medical facility, food, food processing facilities or equipment, soil, water or other target. Such applications may be performed at any density of functionalized phages of the invention, for example, 10³ to 10¹³ PFU, 1 to 100 billion of phages, or 500-1,000 nanogram functionalized phages of the invention per ml.

Following application, and prior to energetic treatment, the phage may be given a period of time to bind target bacterial cells, for example, a time interval in the range of minutes to hours, for example, in the range 5-100 minutes, for example 10-20 minutes.

In some implementations, such as topical administration, a wash step may be performed to remove unbound phage from the target site to prevent thermal damage to non-target cells by free phage. The wash may be by sterile water, saline, buffer, or other solution.

Next, energy, for example, light energy, is applied at suitable intensity, wavelength, and duration to create heat by the plasmonic excitation of nanoparticles aggregated by phage adsorption to target cells. In some implementations, the nanoparticles are configured for excitation at wavelengths in the near infrared, for example, light having a wavelength between 650 to 2,500 nm, for example, 650 to 1,350 nm. For example, gold nanorods of width between 5-15 nm and length of 10-50 nm will generally be excitable within these wavelengths. Advantageously, tissues are highly transmissive to near infrared wavelengths, enabling cell killing deep within tissues, at centimeter scale-depths. This enables the use of injected (e.g. subcutaneous or intravenously injected) functionalized phages to treat infections internally. Effective treatments will heat target bacteria to lethal temperatures, for example, in the range of 40° C.-70° C. Depending on the conditions, light applications may have a duration of seconds to minutes, for example, 30 seconds to 30 minutes, for example, illumination times between 1 and 15 minutes, for example, 5-10 minutes.

The exogenous energy source may comprise any light source sufficient for excitation of the selected nanoparticles of the phage, such as a laser or LED light sources. In some embodiments, the light source is a handheld light source. In some embodiments, the light source is an endoscopic light delivery system, such as a catheter-mounted fiberoptic instrument. Exemplary light sources for excitation in the near infrared wavelengths include infrared lasers such as Ti:sapphire lasers or phosphor conversion LED systems. Exemplary systems include those that deliver in NIR region (e.g. 700-1600 nm), with power ranging from 0.5-4.0 W cm⁻². For example, one example of such as system is the OMNILUX™ (GlobalMed Technologies).

For phage comprising non-plasmonic functional nanoparticles, suitable treatments may be administered in place of or in combination with illumination in order to activate or release functional agents conjugated to the phage. For example, the application of magnetic fields, electric fields, heat, ultrasonic waves, and other forms of energy may be applied, for example, to induce lethal heat or the release of cell killing agents. For example, in some implementations, the modified phage are functionalized with responsive materials and encapsulated, wherein the application of a stimulating treatment, such as magnetic field, electric field or heat will induce release the encapsulated. To achieve this goal, the nanoparticles (gold nanorods) can be decorated with a layer of stimulus-responsive polymers or other materials. In another embodiment, ultrasound is used to break phage-bound nanoparticles containing drugs, such as antibiotics, adapted from similar methods for chemotherapy applications.

Bacterial Detection Methods. In one aspect, the scope of the invention is directed to the use of the nanoparticle-functionalized phages to detect target bacteria. In these methods, plasmon resonance-induced optical effects which are responsive to the aggregation of the nanoparticles on host bacteria, are measured to determine the presence and/or abundance of the target bacteria.

The bacterial detection methods of the invention provide the art with an assay that can detect target cells rapidly, with great sensitivity and specificity. For example, in some implementations, as few as about 100 bacterial cells may be resolved, with minimal noise introduced by the presence of non-target bacterial species. The assay can be performed over a period of minutes. Remarkably, the assay is robust even in challenging media, for example, in complex samples such as seawater and human serum.

In a first implementation, bacterial detection is achieved by the use of phages functionalized with plasmonic nanoparticles, in a method comprising the following steps:
plasmonic nanoparticle-functionalized phages having specificity for a selected target bacteria are applied to a sample;
the sample and functionalized phages are incubated for a sufficient period of time for the functionalized phage to adsorb to target bacterial cells, if present in the sample;
the sample is illuminated with light energy sufficient to induce plasmon resonance excitation in the nanoparticles;
concurrently with the illumination step, a selected optical signal is measured wherein such optical signal is responsive to plasmon resonance excitation of aggregated nanoparticles; and
by the use of an established relationship between optical signal value and target bacteria presence or abundance, the measured value of the optical signal is used to determine the presence or abundance of the target bacteria in the sample.

In a second, alternative implementation, a processing step is performed to isolate and concentrate the bacterial cells;
the sample and functionalized phages are incubated for a sufficient period of time for the functionalized phage to adsorb to target bacterial cells, if present in the sample;
a treatment is applied to isolate bacterial cells, including any phage-adsorbed target bacterial cells, from the sample and a solution comprising the isolated bacterial cells is made;
the sample, or, if a solution of isolated bacterial cells is formed, the solution is illuminated with light energy sufficient to induce plasmon resonance excitation in the nanoparticles;
concurrently with the illumination step, a selected optical signal is measured wherein such optical signal is responsive to plasmon resonance excitation of aggregated nanoparticles; and
by the use of an established relationship between optical signal value and target bacteria presence or abundance, the measured value of the optical signal is used to determine the presence or abundance of the target bacteria in the sample.

In another implementation, bacterial detection is achieved by the use of phages competent to bind plasmonic nanoparticles, in a method comprising the following steps:
phages competent to bind a selected plasmonic nanoparticle type, wherein such phages have specificity for a selected target bacteria, are applied to a sample;
the sample and applied phages are incubated for a sufficient period of time for the phages to adsorb to target bacterial cells, if present in the sample;
nanoparticles of the selected type are applied to the sample under conditions that facilitate conjugation of the nanoparticles to compatible moieties on the phage;
the sample is illuminated with light energy sufficient to induce plasmon resonance excitation in the nanoparticles;
concurrently with the illumination step, a selected optical signal is measured wherein such optical signal is responsive to plasmon resonance excitation of aggregated nanoparticles; and
by the use of an established relationship between optical signal value and target bacteria presence or abundance, the measured value of the optical signal is used to determine the presence or abundance of the target bacteria in the sample.
   In this implementation, the phages competent to conjugate plasmonic nanoparticles may comprise thiolated phage, as described herein. In a variation of this process, an intermediate processing step is performed to isolate and concentrate the bacteria in the sample:
phages competent to bind a selected plasmonic nanoparticle type, wherein such phages have specificity for a selected target bacteria, are applied to a sample;
the sample and applied phages are incubated for a sufficient period of time for the phages to adsorb to target bacterial cells, if present in the sample;
a treatment is applied to isolate bacterial cells, including any phage adsorbed bacterial cells, from the sample and a solution comprising the isolated bacterial cells is made;
nanoparticles of the selected type are applied to the solution under conditions that facilitate conjugation of the nanoparticles to compatible moieties on the phage;
the solution is illuminated with light energy sufficient to induce plasmon resonance excitation in the nanoparticles;
concurrently with the illumination step, a selected optical signal is measured wherein such optical signal is responsive to plasmon resonance excitation of aggregated nanoparticles; and
by the use of an established relationship between optical signal value and target bacteria presence or abundance, the measured value of the optical signal is used to determine the presence or abundance of the target bacteria in the sample.

The sample may be any sample type desired. In one embodiment, the sample is a clinical sample such as blood, serum, urine, saliva, a throat swab, a wound swab, wound exudate, a biopsy, or any other composition of matter derived from a subject. The subject may be any animal, for example, a human patient, test animal, or veterinary subject. In another implementation, the sample is an environmental sample, for example comprising groundwater, soil or other material wherein target bacteria may be present. In another implementation, the sample is a food or agricultural sample, for example, comprising animal parts, animal waste, or foodstuffs. In one implementation, the sample may comprise cultured cells, for example, wherein material isolated from a biological or environmental sample enumerated above is provided with growth medium and incubated for a sufficient period of time under suitable conditions to propagate putatively present bacterial present in the sample. The culture step provides a means of amplifying the signal for low-abundance target bacteria.

The phages may be introduced to sample by any means, for example by pouring, mixing, or otherwise exposing sample material to the phage. Phage solutions may comprise phage in buffer, growth media, or preservatives. Phage concentrations in the solution may vary, for example, in one embodiment, being in the range of 10¹⁰-10¹³, for example, 10¹²-10¹³ phage particles per ml. For example, an aliquot of phage solution comprising in the range of 10¹⁰ to 10¹³ phages may be applied to the sample.

In the incubation step performed, the admixture of sample and phage is incubated under suitable conditions and timing for phage adsorption to any target bacteria present in the sample. For example, in some embodiments, the incubation is performed at body temperature, e.g. 37° C., or room temperature, e.g. 20-30° C. Incubation times of, for example, 5-20 minutes may be utilized, for example incubation times of 15-45 minutes.

During the incubation step, phage will adsorb to target bacteria, if present in the sample, for example, in some cases, the adsorption being wholly or partially mediated by RBPs of the phage. Non-specific interactions may further stabilize phage adsorption to the target cell, including adsorption and tail fiber mediated interactions with bacterial elements, sometimes augmented by phage enzymatic elements, for example, peptidoglycan degrading enzymes.

Following the incubation step, typically it is advantageous to perform an isolation step to concentrate the bacterial cells. The isolation step encompasses any process that isolates phage-adsorbed bacterial cells from the sample or reaction mixture. In a simple implementation, cells are isolated from the phage-sample reaction mixture by centrifugation, for example, centrifugation at 2,000-10,000 RPM, for example, centrifugation at 5,000 RPM, for 2-10 minutes may be used. Supernatant is discarded and the isolated bacterial cells pellet are resuspended in a selected solvent such as buffer or water to create a solution.

In the second implementation of the invention, as set forth above, a functionalization step is performed to conjugate the selected nanoparticle to the phage. The selected nanoparticle is applied to the solution, at a concentration sufficient for and under conditions sufficient for conjugation of the nanoparticle to bind or otherwise associate with the phage. In one embodiment, the phage is a thiolated phage comprising a plurality of free thiol groups, and the selected nanoparticle is gold, for example, gold nanorod. In the first implementation of the invention, as set forth above, the phage applied to the sample is already functionalized with the selected nanoparticle, and the conjugation step is omitted.

Next, the detection process is performed. In a first process, light energy is applied to the solution, wherein the light energy is applied at sufficient wavelength, intensity, and duration to induce plasmon resonance optical effects in any nanoparticle aggregates formed by the adsorption of phage to target bacteria. For example, in some implementations, the selected nanoparticle is gold nanorods, and the applied light has a wavelength and intensity sufficient to induce plasmon resonance in the gold nanorods.

Simultaneously, one or more optical properties of the solution is measured. The one or more optical properties of the sample or solution may be selected from light absorption (e.g., absorbance spectroscopy), a shift in peak absorbance wavelength, static or dynamic light scattering, light refraction, fluorescence, or colormetric analysis. In one embodiment, the optical property of the sample or solution is peak absorbance wavelength, for example, when absorbance of the sample or solution is measured across a range of wavelengths, the range of wavelengths being selected to be responsive to the selected type of nanoparticle and illumination. For example, absorbance across a range of wavelengths in the range of 100-2,000 nm may be performed, for example, 200-1,000 nm, for example, 400-800 nm. These signals are responsive to plasmon resonance in the solution, which is produced by the excited nanoparticles and is highly responsive to the abundance of nanoparticles, for example, by aggregates formed by phage adsorption to target bacteria.

Finally, the measured signal is compared to a previously established relationship between signal value and target bacteria abundance to calculate the abundance of target bacteria in the sample, for example, in units such as colony forming units (CFU), or number of cells per ml, etc.

For example, an equation or standard curve relating measured optical property values to target bacteria abundance may be used. In one embodiment, the relationship between bacterial abundance and absorbance at a selected wavelength is used. In one embodiment, the comparison means is a color chart wherein the user may compare the color of the resuspended bacterial cell solution against a visual aid comprising a range of depicted colors, wherein bacterial abundance values are associated with each color.

The optical signal measurements may be achieved by any number of imaging modalities, including by ultraviolet visible spectroscopy, infrared and near-infrared spectroscopy, two-photon enhanced luminescence, dark-field mode microscopy, transmission electron microscopy, optical coherence tomography, photoacoustic tomography, or other imaging modalities. Detector such as Fiber-coupled optical detectors or simple charge-coupled detector (CCD) cameras may be used to detect the light transmission effects of the nanoparticle aggregates. Advantageously, in some implementations, a colormetric change is sufficiently strong that it can be discerned by eye.

The plasmonic signals generated by nanoparticle aggregation enable qualitative detection or highly sensitive quantification of target cell abundance. In one embodiment, the bound phage detection step is a qualitative assay wherein the presence or absence of target bacteria in the sample is determined. In one embodiment, the bound phage detection step is a quantitative assay wherein the abundance of target bacteria in the sample is determined.

In one embodiment, the selected nanoparticle is gold and the aggregation of gold nanoparticles changes the color of the isolated bacterial cell solution from pink (no bound target bacteria) to purple, with increasingly dark purple color with increasing target bacteria abundance. Such facile determination of target presence and abundance is especially advantageous for point-of-care applications, wherein practitioners can determine the presence of the target bacteria in a sample without the need for specialized equipment.

The detection methods of the invention may be applied in various contexts and applications. In one implementation, the scope of the invention encompasses diagnostic methods for determining the presence of a target bacteria type in a sample, wherein the engineered phage of the invention is applied to the sample and phage-bound bacterial cells are detected/quantified, as described in the foregoing sections. The method may further encompass the selection and application of a suitable therapeutic treatment if the target bacteria is detected. For example, if the target species comprises a species that is resistant to certain antibiotics and treatable by other antibiotics, the proper antibiotic may be selected if the target bacteria is detected.

Diagnostic Methods and Related Treatment Methods. In some embodiments, the scope of the invention encompasses a method of diagnosing a bacterial infection, encompassing the application of one of the detection methods described herein to determine the presence or absence, or abundance over a selected threshold, of a selected target bacteria. If the bacteria is detected (present, or present at an abundance exceeding a selected threshold), an appropriate treatment may be selected and administered based on the target strain being determined to be present. In one embodiment, the scope of the invention encompasses a kit, such as a point of care diagnostic kit, comprising functionalized phage of the invention, or a combination of phage competent to conjugate a selected nanoparticle and the selected nanoparticle, in combination with items such as reagents, cuvettes, containers, or other tools for applying the phage to sample and measuring target bacteria abundance, for example, color cards, instructions, or software (for example, embodying standard curves) or other means of interpreting color or other measurable properties of the phage after its application to the sample.

Detection By Non-Plasmonic Functional Nanoparticles. In implementations where the phage is functionalized with non-plasmonic nanoparticles that enable detection, a suitable imaging process may be performed to quantify the abundance of the nanoparticles. For example, depending on the type of signal generated by the nanoparticles, microscopy, fluorescence measurements, magnetic scanning, or other detection modalities may be employed to quantify nanoparticle abundance. Measured values are compared to standard curves or like relationships that relate measured signal to bacterial abundance. This process may be performed in place of or in combination with the use of plasmonic nanoparticle to detect the target species.

### Exemplary Embodiments

In various embodiments, the scope of the invention encompasses a functionalized phage, comprising a phage comprising a one or more targeting moieties which confer specificity for a selected bacteria type; and wherein a plurality of plasmonic nanoparticles have been conjugated to the phage: wherein the targeting moiety comprises any peptide, protein, or composition of matter that facilitates phage adsorption, binding, or other selective association with a target bacteria; wherein in some embodiments, the one or more targeting moieties comprises a phage receptor-binding protein; in some embodiments, the receptor binding protein is a heterologous receptor binding protein derived from another phage type and the phage has been genetically modified to express such receptor binding protein; in some embodiments, the RBP is an engineered sequence comprising a hybrid, synthetic, or otherwise non-natural RBP sequences; in in some embodiments, the targeting moiety is a protein or polypeptide with specificity for eukaryotic microbes such as yeast, in some embodiments, the targeting polypeptide is a receptor having a complementary ligand on a target cell surface, such as an extracellular protein domain, a carbohydrate moiety, or a bacterial lipid, in embodiments, the targeting polypeptide is a sequence derived from the antigen-binding region of an antibody having high affinity for target cell epitopes: in some embodiments the targeting moieties are expressed as elements of (or conjugated to) tail fibers, tail spikes, baseplate proteins, coat proteins, or capsid proteins: in some embodiments, the targeting moieties confer specificity to target bacteria that are gram negative bacteria and/or antibiotic-resistant bacteria:

in some embodiments the plasmonic nanoparticles are responsive to light of wavelengths between 650 and 2,500 nm; wherein in some embodiments, the nanoparticles comprise a material selected from the group consisting of gold, silver, copper, aluminum, iron, iron oxides, zinc, cadmium, lanthanum, lead, tin, mercury, an alloy of the foregoing, functionalized carbon nanotubes or graphene, and a plasmonic organoometallic composition; in some embodiments, the plasmonic nanoparticle comprises gold or silver comprise nanorods, nanostars, nanospheres, or nanoprisms, nanofiliaments, nanotubes, triangular prisms, nanocubes, or nanocages:
in certain embodiments, the plasmonic nanoparticles comprise nanorods, wherein the nanorods have an aspect ratio, measured as the length to width, of 2:1 to 10:1, in some embodiments, the aspect ratio being 3:1, 4:1 or 5:1, or within plus or minus 50% of such values:
in some embodiments, the plasmonic nanoparticles comprise nanorods having a of width between 5-15 nm and length of 10-50 nm:
wherein in some embodiments, the plasmonic nanoparticles are conjugated by bonds with amino acids or other functional handles on the capsid protein; coat protein; in some embodiments the plasmonic nanoparticles are conjugated to coat proteins by bonds formed with thiol groups, in some embodiments the coat protein is g8p or homolgous coat protein: in some embodiments the phage is selected from the group consisting of M13, MS2, T4, T5, T7, K1F, K11, fd, f1 or SP6:
in some embodiments the phage is formulated in a pharmaceutical composition; the pharmaceutical composition may comprise phage admixed in any number of pharmaceutically acceptable carriers, including buffers, excipients, preservatives, diluents, encapsulating materials, releasing agents, coating agents, antioxidants, and other materials, biocompatible polymers like polyethylene glycol or polylactic acid to form hydrogel/microgels:
in some embodiments, the phage is functionalized with non-plasmonic functional nanoparticles, in some embodiments, the functional nanoparticles being quantum dots, magnetic nanoparticles; drug binding or drug-loaded nanoparticles dendrimers, hydrogels, carbon nanotubes, liposomes, vesicles, caging molecules, and drug delivery particles.

In one embodiment, the functionalized phage is a phage selected from the group consisting of M13, MS2, T4, T5, T7, K1F, K11, fd, f1 or SP6; the one or more targeting moieties comprises a heterologous receptor binding protein expressed by the phage and derived from another phage type; the phage is functionalized with a plurality of plasmonic nanoparticles comprising gold or silver nanorods having an aspect ratio, measured as the length to width ratio, of 2:1 to 10:1;

wherein the nanorods have a width between 5-15 nm and a length between 10-50 nm; and
wherein the nanoparticles are conjugated to the phage by bonds formed with thiolated coat proteins.

In some embodiments, the scope of the invention encompasses a functionalized phage, for use in a method of killing bacterial cells in a subject; wherein the method comprises administering the phage of any of claims 1-15 to the subject, wherein phage adsorption to bacterial cells creates aggregated nanoparticles; and applying light of a suitable wavelength and intensity to induce plasmon resonance in the aggregated nanoparticles; wherein localized non-radiative heating produced by the plasmon resonance kills the bacterial cells to which the phages are adsorbed and destroys the phages. In some embodiments, the administration is intravenous, intra-arterial, intraperitoneal, intrapulmonary, oral, inhalation, intravesicular, intramuscular, intra-tracheal, subcutaneous, transmucosal, or transdermal: in some embodiments the phage is applied to a wound or abscess, to a subject at risk of or suffering from a bacterial infection; in some embodiments aim the applied light is of a wavelength between 650 and 2,500 nm.

In some embodiments, the scope of the invention encompasses the use of the functionalized phages of the invention to detect target bacteria in a sample, comprising: applying a plurality of functionalized phages to a sample, wherein the selected bacteria type is of a type the phage have specificity for by the one or more targeting moieties of such phage: incubating the sample for a sufficient period of time for the functionalized phage to adsorb to bacterial cells of the selected type, if present in the sample; illuminating the sample with light energy sufficient to induce plasmon resonance excitation in the nanoparticles; concurrently with the illumination step, measuring a selected optical signal wherein such optical signal is responsive to plasmon resonance excitation by nanoparticles aggregated by adsorption to bacterial cells of the selected type; and by the use of an established relationship between optical signal value and the presence or abundance of bacterial cells of the selected type, the measured value of the optical signal is used to determine the presence or abundance of cells of the selected bacteria type in the sample:

in some embodiments the optical signal is any signal or property that is responsive to plasmon resonance of measured materials, is responsive to the abundance of nanoparticles, is responsive to signals by aggregates formed by phage adsorption to target bacteria, including: light absorption, a shift in peak absorbance wavelength, static or dynamic light scattering, light refraction, fluorescence, a colormetric property, in one embodiment, the optical property is peak absorbance wavelength, for example, when absorbance of the sample or solution is measured across a range of wavelengths, the range of wavelengths being selected to be responsive to the selected type of nanoparticle and illumination; in some embodiments the wavelengths being in the range of 100-2,000 nm, 200-1,000 nm, or 400-800 nm;
in some embodiments the signal is measured by visible light spectroscopy, ultraviolet visible spectroscopy, infrared or near-infrared spectroscopy, two-photon enhanced luminescence, dark-field mode microscopy, transmission electron microscopy, optical coherence tomography, photoacoustic tomography, or other imaging modalities:
wherein the measured signal is compared to a previously established relationship between signal value and target bacteria abundance to calculate the abundance of target bacteria in the sample, such as an equation or standard curve relating measured optical property values to target bacteria abundance; in some embodiments, the comparison means is a color chart or like tool wherein the user may compare the color of the resuspended bacterial cell solution against a visual aid comprising a range of depicted colors, wherein bacterial abundance values are associated with each color.

In a variation of the detection method, the method comprises the additional step of isolating or concentrating bacterial cells from the sample following incubation, and, subsequently applying the illumination and measurement steps to a solution of isolated or concentrated bacterial cells in place of the sample.

In an alternative detection method: bacterial cells of a selected type in a sample, are detected by a process comprising: applying a plurality of phages to the sample, wherein the phage have specificity for the selected bacteria type and are competent for functionalization with a selected plasmonic nanoparticle; incubating the sample for a sufficient period of time for the applied phages to adsorb to bacterial cells of the selected type, if present in the sample; applying plasmonic nanoparticles of the selected type to the sample under conditions that facilitate conjugation of the plasmonic nanoparticles to compatible moieties on the phage; illuminating the sample with light energy sufficient to induce plasmon resonance excitation in the plasmonic nanoparticles; concurrently with the illumination step, measuring a selected optical signal wherein such optical signal is responsive to plasmon resonance excitation by nanoparticles aggregated by adsorption to bacterial cells of the selected type; and by the use of an established relationship between optical signal value and the presence or abundance of bacterial cells of the selected type, the measured value of the optical signal is used to determine the presence or abundance of cells of the selected bacteria type in the sample:

in this alternative method, the phage competent to conjugate nanoparticles may be a phage that has thiolated coat proteins or other activated or activable functionalization sites for forming bonds with the selected plasmonic nanoparticle: the plasmonic nanoparticles may comprise nanorods, nanostars, nanospheres, or nanoprisms, nanofiliaments, nanotubes, triangular prisms, nanocubes, or nanocages; the nanoparticles may be responsive to light of wavelengths between 650 and 2,500 nm; may comprise a material selected from the group consisting of gold, silver, copper, aluminum, iron, iron oxides, zinc, cadmium, lanthanum, lead, tin, mercury, an alloy of the foregoing, functionalized carbon nanotubes or graphene, and a plasmonic organoometallic composition; may comprise nanorods, nanostars, nanospheres, or nanoprisms; the plasmonic nanoparticles may have an aspect ratio, measured as the length to width, of 2:1 to 20:1, or greater, in some embodiments being between 2:1 to 10:, in some embodiments being 3:1 to 5:1; in some embodiments, the plasmonic nanoparticles comprise nanorods having a of width between 5-15 nm and length of 10-50 nm:
in some embodiments, the phage competent to be functionalized with the plasmonic nanoparticles may be a phage selected from the group consisting of M13, MS2, T4, T5, T7, K1F, K11, fd, f1 or SP6; in some embodiments, the phage has specificity for the selected bacteria type by expression of a receptor binding protein or other targeting moiety which it has been genetically engineered to express;
in some embodiments, the optical signal is peak absorbance or color.

In an exemplary embodiment, the phage competent to conjugate nanoparticles comprises a phage is selected from the group consisting of M13, MS2, T4, T5, T7, K1F, K11, fd, f1 or SP6; one or more targeting moieties comprises a heterologous receptor binding protein expressed by the phage and derived from another phage type; and comprises thiolated coat proteins. In an exemplary embodiment, the plasmonic nanoparticles with which the phage is functionalized comprise gold or silver nanorods having an aspect ratio, measured as the length to width ratio, of 2:1 to 10:1; wherein the nanorods have a width between 5-15 nm and a length between 10-50 nm; and wherein the nanoparticles may be conjugated to the phage by bonds formed with thiolated coat proteins.

In an alternative variation of the method, the method comprises the additional step of isolating or concentrating bacterial cells from the sample following incubation, and applying the illumination and measurement steps to a solution of isolated or concentrated bacterial cells in place of the sample.

In the detection methods of the invention, in some embodiments, the sample is selected from the group consisting of a clinical sample an environmental sample, a food or agricultural sample, and cultured cells.

### EXAMPLES

Example 1. Photothermal ablation of specific bacterial species using gold nanorods targeted by chimeric phages. In this demonstration, phages were conjugated to gold nanorods, creating a reagent that can be destroyed upon use (termed ‘phanorods’). Chimeric phages were engineered to attach specifically to several gram-negative organisms, including the human pathogens E. coli, Pseudomonas aeruginosa, and Vibrio cholerae, and the plant pathogen Xanthomonas campestris. The bioconjugated phanorods could selectively target and kill specific bacterial cells using photothermal ablation. Following excitation by near-infrared light, gold nanorods release energy through non-radiative decay pathways, locally generating heat that efficiently kills targeted bacterial cells. Specificity was highlighted in the context of a P. aeruginosa biofilm, in which phanorod irradiation killed bacterial cells while causing minimal damage to epithelial cells. Local temperature and viscosity measurements revealed highly localized and selective ablation of the bacteria. Irradiation of the phanorods also destroyed the phages, preventing replication and reducing potential risks of traditional phage therapy while enabling control over dosing. The phanorod strategy integrates the highly evolved targeting strategies of phages with the photothermal properties of gold nanorods, creating a well-controlled platform for systematic killing of bacterial cells.

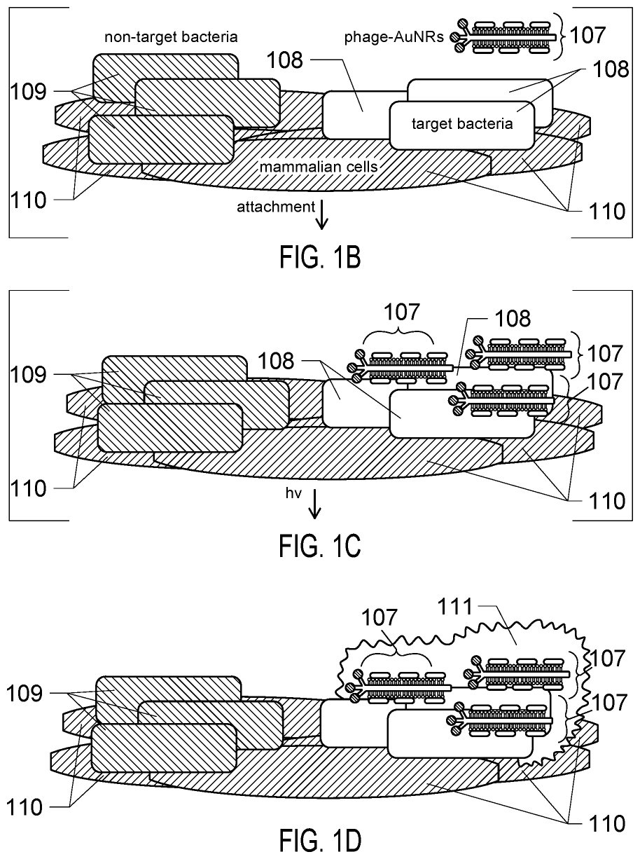

Construction of phage-AuNR bioconjugates. The gold nanorods were synthesized following a typical seed-mediated protocol, resulting in uniform particles with an average aspect ratio of 3.8 (average length=53.2 nm; average width=13.7 nm). The UV-vis spectrum of the AuNRs demonstrated transverse and longitudinal absorption peaks at 526 nm and 800 nm, respectively (FIG. 2). The capsid of M13KE phage was modified with SATP to introduce thiol groups to primary amines. Thiolation resulted in new FTIR signals at 1736 cm⁻¹ and 2558 cm⁻¹, corresponding to C═O (from SATP) and S—H (thiol group) stretching, respectively, indicating successful modification of the virions, designated M13KE-SH. The overall morphology of the phage, assessed by TEM, was not affected by thiolation.

M13KE-SH was conjugated to AuNRs by formation of gold-sulfur bonds at room temperature in Tris buffer (pH 3.0). Interaction between AuNRs and phages during bioconjugation was promoted by the positive charge from trace CTAB on the AuNRs=(ζ21.9 mV) and the negatively charged capsid protein of phage particles=−44.3 mV). The formation of Au—S bonds in the bioconjugates was confirmed by X-ray photoelectron spectroscopy. The binding energies of the sulfur electrons (S_{2p3/2} and S_{2p1/2}) in thiol groups were 163.6 and 164.8 eV, respectively; these peaks were present in both M13KE-SH and the M13KE-AuNR bioconjugates. However, additional electron binding energies (S_{2p3/2} and S_{2p1/2}) from the Au—S bond appear at 161.8 and 163.0 eV, respectively, in M13KE-AuNR but not in M13KE-SH. In particular, the S_{2p3/2} peak at 161.8 eV can be readily identified as a new signal, confirming successful conjugation of the AuNRs to the phage particles. Trace CTAB was then replaced by ligand exchange with carboxylated PEG (HS-PEG-COOH) after bioconjugation. Formation of bioconjugates was also confirmed by TEM, which indicated ˜10 AuNRs per phage. Another approach to estimate the ratio of AuNRs to phage particles is inductively coupled plasma mass spectrometry (ICP-MS) to measure the amount of AuNRs and quantitative PCR to measure the amount of phage in a sample; this approach indicated ˜20 AuNRs per phage. Thus an estimate of the number of AuNRs conjugated per phage particle is roughly 15.

The UV-Vis spectrum of the bioconjugates indicates a red-shift of ˜10 nm compared to AuNRs alone. The negatively charged surface of HS-PEG-COOH-modified M13KE-AuNR=(ζ=−28.8 mV) should reduce non-specific binding to bacteria considering the negatively charged cell surface=(ζ=−8.88 mV). TEM demonstrated that while HS-PEG-COOH-modified AuNRs do not attach to E. coli cells in the presence of non-conjugated M13KE, phage-AuNRs attach to E. coli cells, as expected. To further confirm that the M13KE-AuNR bioconjugates retain the ability to interact with E. coli, M13KE-AuNRs were labeled with a fluorescent dye using fluorescein-5-maleimide (FITC) through thiol-maleimide click chemistry, as M13KE-AuNRs contained free thiols according to the XPS spectrum. The FITC-labeled M13KE-AuNRs were incubated with E. coli expressing a cyan-fluorescent protein and visualized by confocal microscopy, which verified close proximity of FITC and cyan fluorescence.

Having verified the method with M13KE, AuNR bioconjugates were also prepared with chimeric phages M13-g3p(If1), M13-g3p(Pf1), M13-g3p(ϕLf), M13-g3p(ϕXv), and M13-g3p(CTXϕ), targeting E. coli (I⁺), P. aeruginosa, X. campestris pv. vesicatoria, X. campestris pv. campestris, and V. cholerae, respectively (31) (Table 1).

TABLE 1
Chimeric phage bioconjugates and targeted bacterial species.

| | Source of | Designation of |
|---|---|---|
| Bacterial target strain | RBP | bioconjugates |
| E. coli (F⁺), ER2738 | wild-type M13 | M13KE-AuNR |
| V. cholerae 0395 | CTXϕ | M13-g3p(CTχϕ)-AuNR |
| E. coli (I⁺), (Migula) | Iϕ1 | M13-g3p(If1)-AuNR |
| Castellani and Chalmers | | |
| X. campestris | ϕLf | M13-g3p(ϕLf)-AuNR |
| (pv. campestris) | | |
| X. campestris | ϕχv | M13-g3p(ϕXv)-AuNR |
| (pv. vesicatoria) | | |
| P. aeruginosa (Schroeter) | Pf1 | M13-g3p(Pf1)-AuNR |
| Migula | | |

Detection of specific bacterial species by phage-AuNRs. In Example 2, thiolated phages to target aggregation of gold nanospheres (AuNPs) causes a red-shift of localized surface plasmon resonance peaks in the UV-vis spectrum. Abundant thiol groups were incorporated on carboxylates of the phage capsid (with 3 or more solvent-accessible residues on each g8p protein) to induce aggregation of gold nanoparticles, and removal of free thiolated phage was required to remove background signal. In this example, the level of thiolation of the phage was reduced by using amines for bioconjugation, of which there is only one solvent-accessible residue (at the N-terminus) of each g8p coat protein. The phage-AuNRs synthesized here did not aggregate detectably in the absence of cells, simplifying the detection protocol to single-step addition of the bioconjugates to the cell sample in appropriate solution. E. coli ER2738 was suspended at varying concentrations in MilliQ water and incubated with M13KE-AuNRs for 30 min. Consistent with prior results using AuNPs, a red-shift and broadening of LSPR peaks of the AuNRs was observed in the presence of ≥10² bacterial cells (FIG. 2), demonstrating the sensitivity of bacterial detection using phage-AuNRs.

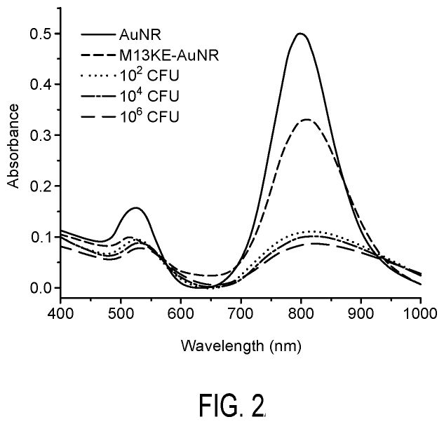

Five chimeric phages recognizing other Gram-negative bacterial strains (I⁺ E. coli, P. aeruginosa, V. cholerae, and two strains of X. campestris) were propagated in E. coli cells and functionalized with AuNRs as described above. As seen with M13KE-AuNRs targeting E. coli, the sensitivity of detection for these other strains was ˜10² CFU using the respective chimeric phage-AuNRs (FIG. 3A).

To verify the specificity of each of the six phage-AuNRs for its respective host, each phage-AuNR was incubated with the other bacterial strains. For each phage-AuNR, no shift or broadening of the LSPR peaks appeared when non-host strains were added, indicating little cross-reactivity among the tested group of Gram-negative organisms. The detection assay was also performed in a mixture of the host strains, and no change of the LSPR peaks was observed unless the heterogeneous mixture contained the targeted host cells (FIG. 3B). These results confirm the ability of the chimeric phages to target the AuNRs to their particular bacterial host.

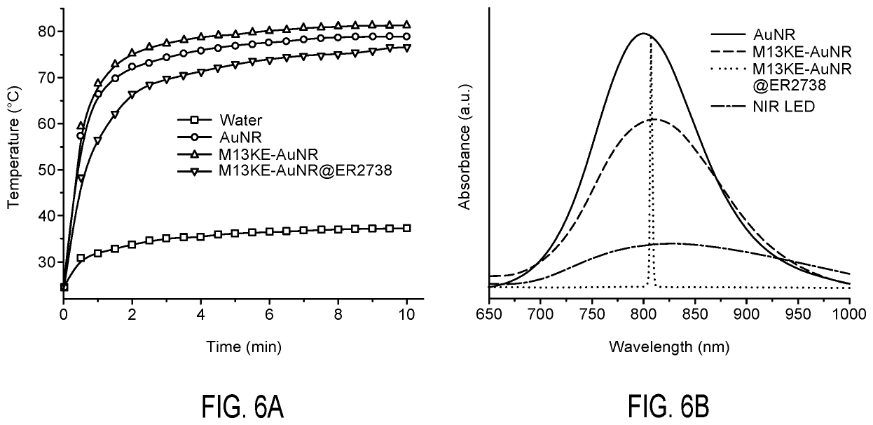

Photothermal ablation of bacterial cells in suspension. The plasmonic resonance of gold nanorods converts light into heat, which can be used to damage and kill cells within a submicron to micron radius. M13KE-AuNRs were irradiated by a near-infrared laser (peak at 808 nm) for 10 min and the bulk temperature of the solution was measured by a thermocouple (FIG. 6A). Some heating (from 24° C. to 37° C.) occurred due to irradiation alone, but solutions containing AuNRs (equivalent to 3.3 nM AuNRs), M13KE-AuNR (equivalent to 3.3 nM AuNRs and 10¹¹ phages/mL), or M13KE-AuNR mixed with E. coli ER2738 (10⁶ cells/mL), reached temperatures of 77-81° C. The slightly lower temperature achieved when M13KE-AuNRs were mixed with cells may be due to the reduced LSPR absorption of the aggregates at 808 nm. Plating of samples containing M13KE-AuNRs mixed with E. coli ER2738 demonstrated that roughly 50% of bacteria were killed by 3 min, ˜90% of bacteria were killed by 6 min, and no viable bacteria remained after 10 min. Similar results were observed using all six phage-AuNR bioconjugates to kill their respective host bacterial cells. TEM imaging of M13KE-AuNRs mixed with E. coli ER2738 cells and irradiated demonstrated grossly altered cell morphology. A live/dead cell-staining assay further verified bacterial cell death by microscopy.

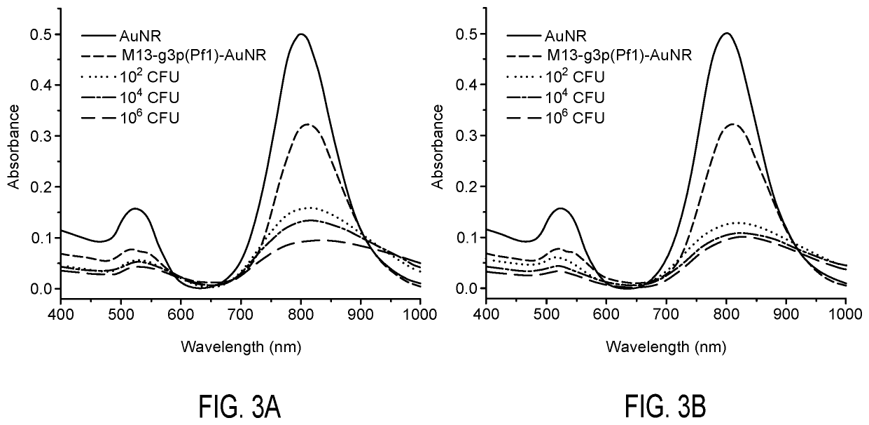

In principle, cell death should occur primarily for the targeted host organism bound by the phage-AuNRs. However, non-targeted cells may also die as the temperature of the bulk solution increases or if they are bound non-specifically by phage-AuNRs. To test the specificity of bacterial cell death, F⁺ E. coli cells (ER2738; host for M13KE) that express cyan fluorescent protein (10⁶ cells/mL) were mixed with F⁻ E. coli cells (BL21; lacks receptor for M13KE) that express citrine fluorescent protein (10⁶ cells/mL), incubated with M13KE-AuNRs (10¹¹ phages/mL), and irradiated to induce photothermal lysis. Samples were plated and viable colonies were counted. The concentration of F⁺⁺E. coli (targeted strain) decreased sharply, with no colony-forming units at 10 min (FIG. 4). In contrast, the concentration of F⁻ E. coli (non-target strain) decreased only slowly, with ˜95% of F⁻ cells surviving at 3 minutes and ˜81% of F⁻ cells surviving after 10 min (FIG. 4). This confirms that the phage-AuNRs distinguished bacterial strains as expected, and selectively killed the targeted cells.

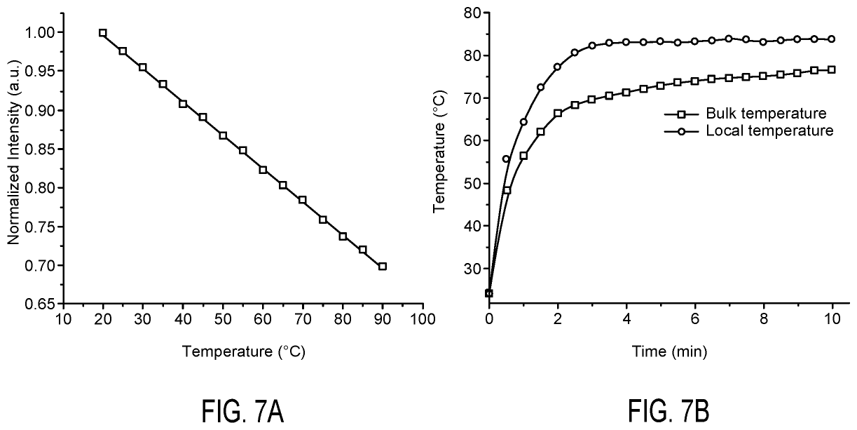

While the bulk temperature increases upon irradiation, binding of phage-AuNRs to bacterial cells should induce localized heating of the cell. To estimate the temperature of the bacteria, E. coli ER2738 were stained with the temperature- and pH-sensitive dye BCECF, whose fluorescence intensity decreases linearly with temperature. The steady-state fluorescence intensity of BCECF was recorded during irradiation of E. coli ER2738 with M13KE-AuNRs. The apparent cell temperature reached a plateau of ˜83° C. after 3 min and rose more quickly than the bulk temperature, being higher than the bulk temperature at all observed times points. The temperature gap between cell temperature and bulk temperature (measured by thermocouple) was observed to be ˜13° C. at 3 min (FIG. 7B). It should be noted that bulk heating observed depends on the concentration of AuNRs as well as heat dissipation properties of the medium and cuvette. The BCECF measurement is also likely to underestimate the true bacterial cell temperature since some dye is also dissolved in the bulk; thus it should be regarded as a lower bound for bacterial cell temperature. In addition, pH is assumed to be constant during irradiation, such that the fluorescence change is attributed to temperature changes. Nevertheless, this measurement validates the qualitative expectation that the targeted cells are heated beyond the level of the bulk solution.

Photothermal ablation of P. aeruginosa in biofilms. Biofilms present an important obstacle to antibiotics and other therapeutic strategies due to the dense macromolecular network and altered physiological state of the biofilm cells. To determine whether photothermal ablation could be effective against bacterial biofilms, P. aeruginosa was grown in a standard biofilm format on glass bottom plates, incubated the biofilm with M13-g3p(Pf1)-AuNRs (10¹³ phages/mL), removed excess liquid by pipetting, and irradiated as described above for 10 min. Live/dead staining of the biofilm showed widespread bacterial cell death, and no colonies were obtained after resuspension and plating of the irradiated biofilm. To gain a rough estimate of the temperature of the biofilm after NIR irradiation, the biofilms were stained with BCECF. To create a calibration curve, a series of fluorescent images was recorded at different temperatures using a confocal microscope, and the pixel intensity (measured by ImageJ) was plotted as a function of temperature. The average bacterial cell temperature captured a few seconds after 10 min of NIR irradiation was estimated to be 84° C. using this calibration curve, indicating similarly efficient heat transfer from the gold nanorods to bacterial cells in the biofilm compared to bulk solution.

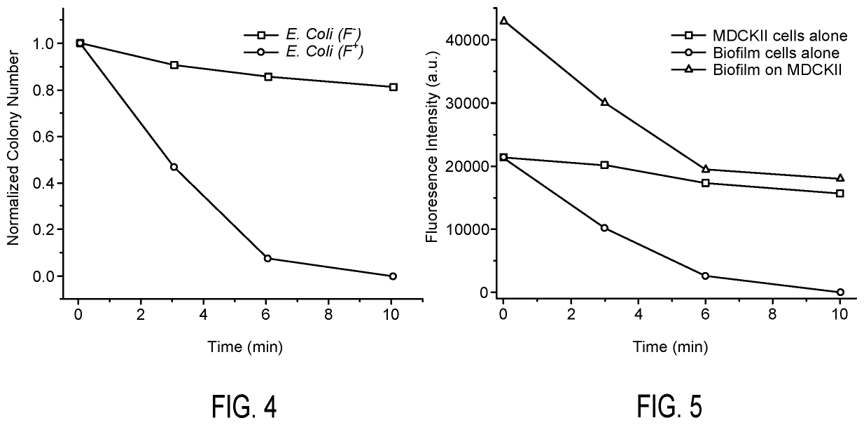

Photothermal ablation of P. aeruginosa biofilm grown on mammalian epithelial cells. While phage-AuNR-mediated heating was effective for killing bacterial cells, it is possible that heat transfer to surrounding mammalian cells could be deleterious. A P. aeruginosa biofilm was gown directly on top of a monolayer of Madin-Darby Canine Kidney II (MDCKII) mammalian epithelial cells and determined the survival of both the bacterial cells and the MDCKII cells after application of M13-g3p(Pf1)-AuNRs (10¹³ phages/mL) with irradiation performed as described above. Microscopy with live/dead staining demonstrated that bacterial cells in the biofilm were killed while MDCKII cells survived, with a majority of bacterial cells dead at 6 min. This result was verified by a PrestoBlue cell viability assay (FIG. 5), which indicated that nearly all bacterial cells were killed after 10 min at the laser power used (3.0 W/cm²). The viability of MDCKII cells without biofilm was reduced to ˜71% by application of M13-g3p(Pf1)-AuNRs and irradiation for 10 min, compared to a control of MDCKII cells without M13-g3p(Pf1)-AuNRs or irradiation. Interestingly, a greater proportion of the MDCKII cells survived (˜84% viability) when covered by the P. aeruginosa biofilm, as determined by PrestoBlue assay after subtracting the fluorescence intensity of the treated (dead) bacterial biofilm. This protective effect could be due to the biofilm adsorbing the M13-g3p(Pf1)-AuNRs, leading to less non-specific binding of the bioconjugates to MDCKII cells, or to a reduction of laser fluence reaching the MDCKII cells due to absorption by the greater number of bioconjugates in the biofilm. Regardless, these results demonstrate survival of the majority of mammalian cells while no bacterial cells survived; optimization of the irradiation protocol may enhance this difference. Furthermore, the phages and bioconjugates themselves (without irradiation) were non-toxic to MDCKII cells in a broad concentration range, as demonstrated by the PrestoBlue cell viability assay.

To further probe the effect of phage-AuNRs on the bacteria and MDCKII cells, the viscosity of cell membranes was characterized using a molecular rotor, a dye whose fluorescence lifetime provides a measurement of local micro-viscosity. The viscosity of the cell membrane is expected to decrease upon intense heating, leading to destruction of membrane order. MDCKII/P. aeruginosa biofilm was stained with the molecular rotor BODIPY C10 and fluorescence lifetime imaging (FLIM) was used to assess membrane viscosities after photothermal treatment. While the MDCKII cells did not exhibit substantial change in fluorescence lifetime after irradiation (2.31±0.17 ns before irradiation; 2.23±0.21 ns after irradiation), the fluorescence lifetime of the dye on P. aeruginosa cells decreased from an average of 2.36±0.12 ns to 0.92±0.09 ns, corresponding to a dramatic drop in viscosity from 296 cP to 38 cP. This finding is consistent with the idea that the phage-AuNRs directly target the bacterial host cells with relatively little damage to other cells.

To verify whether NIR irradiation destroyed the infectious potential of the phages, M13KE-AuNRs were irradiated for 10 minutes and then used to infect E. coli for phage propagation. Putative viral DNA was extracted and assayed by quantitative PCR. No DNA was detected from propagation of the treated sample, confirming that the phages were inactivated during the treatment.

Conclusion. Demonstrated herein is an antibacterial strategy using phages conjugated to gold nanorods (phage-AuNRs, referred to in the following discussion as ‘phanorods’, a portmanteau of ‘phage’ and ‘nanorods’). The phages attach to targeted bacteria, and irradiation of the nanorods by near-infrared light causes localized surface plasmon resonance excitation. This energy is released as heat, destroying the phage as well as bacteria bound to the phage. The phanorod strategy has important advantages over traditional approaches to phage therapy. First, phage therapy suffers from the major difficulty of managing a replicating and evolvable entity. While the evolutionary capacity of phages is advantageous for overcoming bacterial resistance against a phage, evolutionary potential is an important biocontainment concern in practice. Second, nonlinear replication dynamics mean that dosages cannot be easily controlled, which may be problematic if cell lysis releases endotoxins triggering deleterious host responses (e.g., septic shock). Phanorods are destroyed during irradiation, preventing replication and evolution during treatment and enabling control over dosage. Irradiation could also be used to inactivate excess phanorods after use, avoiding negative impacts, such as evolution of resistant organisms, currently associated with antibiotics in the waste stream. Third, evolution of resistance is an important challenge for any antibacterial strategy, including phanorods. However, because the phage is used only for attachment to cells and downstream events (e.g., replication) are not relevant, bacterial mechanisms for resistance should be limited to alterations of the receptor, presenting a smaller mutational target for evolution of resistance. Fourth, phanorods serve simultaneously as diagnosis and cytotoxic reagents, as the change in the LSPR spectrum can be used to recognize bacterial species. Therefore, although there may be situations in which therapy with phages per se is desired (e.g., if exponential replication dynamics are needed), phanorod pharmacokinetics and pharmacodynamics may more closely resemble those of a typical drug rather than a living organism, which would be advantageous for most therapeutic situations. Bacterial biofilms represent a difficult challenge for treatment, as the protective extracellular matrix often inhibits access by antibiotics. However, heat can be transferred without molecular penetration into the biofilm. Effective killing of P. aeruginosa, identified as one of three ‘critical priority’ bacterial pathogens identified by the World Health Organization was demonstrated herein, including killing a P. aeruginosa biofilm grown on epithelial cell culture.

Example 2. Rapid Colorimetric Detection of Bacterial Species through the Capture of Gold Nanoparticles by Chimeric Phages. Members of Inovirus infect a variety of Gram-negative genera of medical and agricultural interest, including Pseudomonas, Xanthomonas, Yersinia, and Neisseria. The RBP, or minor coat protein, pIII, consists of two domains. The N-terminal domain of pIII (encoded by g3p-N) attaches to the primary host receptor (e.g., the F pilus for the Ff phages, such as M13), while the C-terminal domain interacts with a secondary host receptor and aids cell penetration. The replacement of g3p-N by a homologous domain switches attachment specificity to the corresponding host in at least two cases, for example, as described in Heilpern and Waldor, CTX phi Infection of Vibrio cholerae Requires the tolQRA Gene Products. J. Bacteriol. 2000, 182, 1739-1747 and Lin et al., The Adsorption Protein Genes of Xanthomonas campestris Filamentous Phages Determining Host Specificity. J. Bacteriol. 1999, 181, 2465-2471. This was strategy to additional Inovirus members the resulting chimeric phages were thiolated for interaction with AuNPs. This enabled rapid and specific detection of two strains of E. coli, Pseudomonas aeruginosa, Vibrio cholerae, and two strains of the plant pathogen Xanthomonas campestris with a detection limit of ˜100 cells. Here, first is demonstrated the use of thiolated M13 phage to aggregate AuNPs to detect E. coli. Next, the generalization of this strategy is demonstrated using RBPs from five other filamentous phages, allowing the targeting of their respective host species or strain.

The phages were chemically modified by thiolation to generate an interaction with AuNPs. Each major capsid protein (pVIII) of the M13 scaffold contains at least three solvent-accessible carboxylic amino acids at the N-terminus (Glu2, Asp4, and Asp5), which can be potentially modified by EDC chemistry under mild conditions. As a proof of concept, the wild-type M13KE phages were thiolated with cysteamine to detect E. coli ER2738 bacteria. The concentration of chemically incorporated thiol groups was quantified with Ellman's assay, while the concentration of phage particles was determined by real-time PCR. It was estimated that the chemical modification led to the addition of ˜1800 thiol groups per virion. This level is consistent with a substantial fraction of the phage coat being modified (˜2700 copies of pVIII per virion have been reported). Attenuated total reflection Fourier transform infrared (ATR-FTIR) analysis further confirmed the presence of thiol groups on the phage after modification. In addition, the potential of the phage is expected to increase upon thiolation due to the masking of Glu and Asp residues. Indeed, ζ the of unmodified M13KE phage in water was measured to be −44.3 mV, while that of the thiolated M13KE phage was −10.31 mV. These results support the successful functionalization of the phage.

To check the gross morphology of thiolated M13KE virions, their hydrodynamic behavior was measured by dynamic light scattering (DLS). The effective diameter of the wild type phage showed little change after modification. Normal virion morphology and lack of agglomeration was also verified by transmission electron microscopy (TEM). Another potential concern was that thiolation of pIII might interfere with binding to the host cell because there may be solvent-accessible carboxylic amino acids (e.g., Glu2 and Glu5) on pIII. The thiolated M13KE phage was tested for attachment to host cells expressing a cyan-fluorescent protein. The virions were labeled with a fluorescent dye FITC through thiol-maleimide click chemistry and purified. After incubation at room temperature for 30 min to allow for attachment, the sample was visualized by confocal microscopy. The fluorescence of the modified phages was found to be in close proximity to the cell surfaces. Thus, thiolated phages exhibit normal morphology and retain the ability to bind host cells. Furthermore, the dissociation constants (K_{d}) of wild type M13KE and thiolated M13KE for E. coli (F+) were found to be, indicating that thiolation did not substantially perturb attachment to host cells.

Citrate-stabilized AuNPs were synthesized and verified by TEM to have a diameter of ˜4 nm. DLS showed a relatively monodispersed population centered at diameter ˜8 nm. The apparent size difference is reasonable considering the difference in hydration state and the intensity-based weighting of the DLS data. The potential of the AuNPs in water was found to be −45.1 mV, indicating a highly negatively charged surface, intended to stabilize the colloidal particles in solution.

To test the assay principle using thiolated M13KE phage with AuNPs for detection of E. coli, varying concentrations of E. coli ER2738 were diluted into tap water and incubated with the phage for 30 min. The cells (with attached phages) were washed twice and then resuspended in a solution containing AuNPs. In the absence of bacteria or in the presence of unmodified M13KE, a red solution is obtained, consistent with the color of the un-aggregated AuNPs in solution. The aggregation of AuNPs on thiolated phage, indicating the presence of E. coli, was observed by a change in the absorbance spectrum, resulting in a purple solution easily observed by the naked eye. This assay can detect as few as 60 CFU cells. Therefore, the limit of detection is on the order of ˜10² CFU, indicating the high sensitivity of the present technique. Similar sensitivity is seen when a more-concentrated solution of AuNPs was used. While this aggregation-based assay is not ideally suited for creating a standard curve with a large dynamic range, a dilution series of a sample could be used to obtain a rough order-of-magnitude estimate of the concentration of a specific bacterial species. In particular, the dilution at which the number of cells becomes less than ˜100 could be identified and used to infer the concentration of the original sample. To characterize the interaction, TEM images were obtained for the mixtures. Large aggregates containing AuNPs and thiolated phages were observed in samples containing thiolated phages and E. coli cells but were absent when unmodified M13KE was used. Attachment of AuNPs to free bacteria was not observed by TEM, consistent with electrostatic repulsion given the negative zeta potential of AuNPs and E. coli (ζ=−8.88 mV, measured here). The phages are also negatively charged (ζ=−10.31 mV, measured for thiolated M13KE), so AuNP association with the phages is driven by the Au—S interaction despite electrostatic repulsion. It should be noted that free filamentous phage do not pellet at the centrifugation speeds used to pellet the cells and cell-phage complexes. Overall, in this assay, unbound virions were removed and the AuNPs aggregated on the thiolated phages attached to the host bacteria, resulting in a visible color change.

The robustness of a bacterial detection platform in different media is an important consideration for potential applications. To test this, thiolated M13KE was incubated with E. coli in seawater and human serum with the remaining steps carried out as described above. Incubations in all media yield a detectable colorimetric response to the presence of E. coli. E. coli can survive in seawater for several days, similar to survival in freshwater. The change in absorption spectrum for samples incubated in human serum was less pronounced than that for the different samples of water. Given that human serum contains a complex mixture of proteins and other macromolecules, it is possible that some of these components might interfere with the interaction among bacteria, phage, and AuNPs. Nevertheless, the color change of AuNPs on phages was still visible even in this complex media.

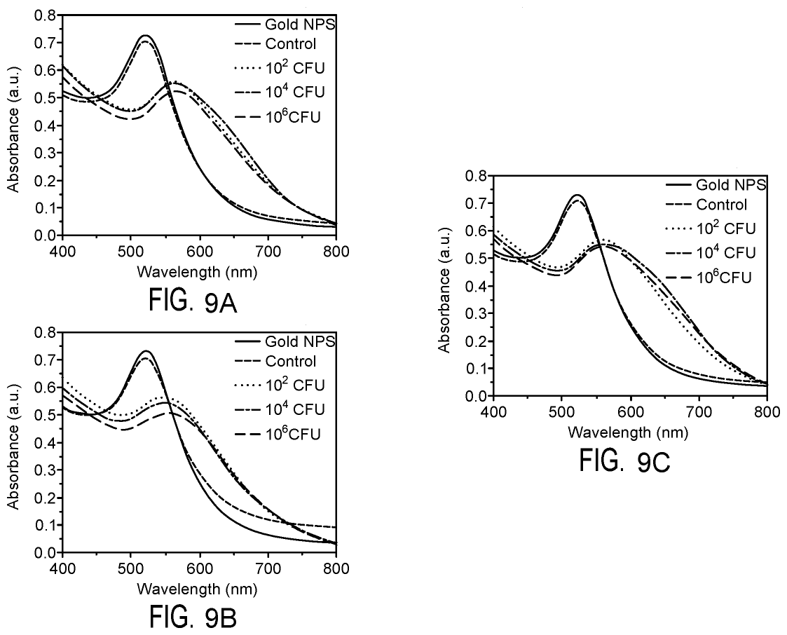

Having validated the technique to detect E. coli, engineered phages capable of recognizing pathogenic bacterial species were made. Chimeric phages using a derivatized M13 genome as a scaffold to display the RBP from five other filamentous phages: CTXφ, If1, φXv, φLf, and Pf1 (Table 1). In each case, the RBP gene of M13 (g3p-N) was replaced by its known or putative homologue from the other phage. The RBP sequences were adjusted for codon bias in E. coli but were used without other optimization. Successful construction was verified by restriction digestion and sequencing. The resulting phages were produced in E. coli cells after transformation. The chimeric phages (M13-g3p(CTXφ), M13-g3p(Pf1), M13-g3p(φLf), M13-g3p(φXv), and M13-g3p(If1)) were thiolated and used to detect their respective host bacteria in tap water, seawater, and human serum. The thiolated chimeric phages showed comparable sensitivity to detect their host bacteria compared to M13KE with F⁺E. coli (FIGS. 9A, 9B, and 9C). The limit of detection in all cases was ˜10² CFU, demonstrating the adaptability of this approach to targeting different bacterial species and strains.

Because the specificity of detection is important for identifying bacteria, each of the six phages (M13KE and the five chimeric phages) was tested for its ability to detect the hosts of the other phages. No shift of SPR peaks in the UV-vis spectrum was observed in any case, indicating little cross-reactivity within the group of Gram-negative organisms tested. This is likely a reflection of the specificity of the source phages themselves. We also tested whether detection by individual phages was affected in a heterogeneous mixture of bacteria [E. coli (F⁺), V. cholerae, and P. aeruginosa]. The red-shift of SPR peaks only occurred when the bacterial mixture contained the host cells targeted by the phage [M13KE, M13-g3p(CTXφ), or M13-g3p(Pf1), respectively], confirming the expected specificity of the phages.

All of the chimeric phages that gave high sensitivity and specificity in the AuNP-based assay without empirical optimization. The assay tolerated tap water and filtered seawater with negligible change. Although human serum decreased the absorbance shift, the assay was still readily interpretable in this media. The tolerance of the assay to different conditions may also reflect the evolutionary history of phages, which have been selected to attach to their hosts in natural, sometimes harsh, environments. The assay itself was performed in less than an hour with a reagent cost of <$1.40 per assay. It is possible to decrease the reagent costs further by use of silver nanoparticles, which give a yellow to orange color change upon aggregation and also interact strongly with thiols. Indeed, AgNPs can were tested and used in analogous fashion in our assay. In addition, a potentially interesting feature of AgNPs is their antimicrobial properties.

Conclusion. Here was demonstrated a platform for the rapid, inexpensive, sensitive, and specific detection of microbial pathogens, based on the phage-bacteria interactions that have evolved in nature. In this design, the RBP of a foreign phage was displayed on an M13 scaffold, creating chimeric phages to bind different host bacteria. The phages were further chemically modified to interact with AuNPs, bridging the target bacteria to the AuNPs, which act as a signal amplifier, as aggregation of the AuNPs causes a visible shift in SPR absorbance. The limit of detection (˜100 cells) in the present assay is comparable with other high-sensitivity assays, and might be lowered by using a lower resuspension volume or by addition of a culturing step. No cross-reactivity was detected for the organisms tested here, although specificity likely depends on the characteristics of the phage RBPs. Substantial versatility was demonstrated here, including detection of two human pathogens as well as two strains of a plant pathogen, with no experimental optimization required. This straightforward approach will be useful for detection and identification of bacteria in situations in which time and/or equipment resources are limited.

All patents, patent applications, and publications cited in this specification are herein incorporated by reference to the same extent as if each independent patent application, or publication was specifically and individually indicated to be incorporated by reference. The disclosed embodiments are presented for purposes of illustration and not limitation. While the invention has been described with reference to the described embodiments thereof, it will be appreciated by those of skill in the art that modifications can be made to the structure and elements of the invention without departing from the spirit and scope of the invention as a whole.

1-42. (canceled)

43. A functionalized phage, comprising
a phage comprising a one or more targeting moieties,
wherein each targeting moiety comprises a receptor-binding protein conferring specificity for a selected target bacteria type; and
wherein a plurality of plasmonic nanoparticles have been conjugated to the phage.

44. The functionalized phage of claim 43, wherein
the plasmonic nanoparticles are responsive to light of wavelengths between 650 and 2,500 nm.

45. The functionalized phage of claim 43, wherein
the nanoparticles comprise a material selected from the group consisting of gold, silver, copper, aluminum, iron, iron oxides, zinc, cadmium, lanthanum, lead, tin, mercury, an alloy of the foregoing, functionalized carbon nanotubes, graphene, and a plasmonic organoometallic composition.

46. The functionalized phage of claim 43, wherein
the nanoparticles comprise nanorods, nanostars, nanospheres, or nanoprisms.

47. The functionalized phage of claim 46, wherein
the nanoparticles comprise nanorods, wherein the nanorods have an aspect ratio, measured as the length to width, of 2:1 to 10:1.

48. The functionalized phage of claim 46, wherein
the nanoparticles comprise nanorods having a of width between 5-15 nm and length of 10-50 nm.

49. The functionalized phage of claim 43, wherein
the target bacteria type is a gram negative bacteria and/or a drug-resistant bacteria.

50. The functionalized phage of claim 43, wherein
the phage is selected from the group consisting of M13, MS2, T4, T5, T7, K1F, K11, fd, f1 or SP6.

51. The functionalized phage of claim 43, wherein
the one or more targeting moieties comprises a heterologous receptor binding protein derived from another phage type.

52. A method of killing cells of a selected bacterial type, comprising,
contacting the bacterial cells with a plurality of functionalized phages, wherein each of the functionalized phages comprises
one or more receptor-binding proteins that confers specificity for the selected bacterial type; and
wherein a plurality of plasmonic nanoparticles have been conjugated to the phage;
wherein phage adsorption to the bacterial cells creates aggregated nanoparticles; and
applying light of a suitable wavelength and intensity to induce plasmon resonance in the aggregated nanoparticles;
wherein localized non-radiative heating produced by the plasmon resonance kills the bacterial cells to which the phages are adsorbed and destroys the phages.

53. The method of claim 52, wherein
the bacterial cells are present in a subject and the functionalized phages are administered to the subject in a therapeutically effective amount.

54. The method of claim 53, wherein
the administration is to a wound or abscess.

55. The method of claim 52, wherein
the bacterial cells are present on or in: a material, a surface, a medical instrument, a surface in a medical facility, food, food processing facility or equipment, soil, or water.

56. The method of claim 52, wherein
the plasmonic nanoparticles are responsive to light of wavelengths between 650 and 2,500 nm.

57. The method of claim 52, wherein
the nanoparticles comprise a material selected from the group consisting of gold, silver, copper, aluminum, iron, iron oxides, zinc, cadmium, lanthanum, lead, tin, mercury, an alloy of the foregoing, functionalized carbon nanotubes, graphene, and a plasmonic organoometallic composition.

58. The method of claim 52, wherein
the nanoparticles comprise nanorods, nanostars, nanospheres, or nanoprisms.

59. The method of claim 58, wherein
the nanoparticles comprise nanorods, wherein the nanorods have an aspect ratio, measured as the length to width ratio, of between 2:1 to 10:1.

60. The method of claim 58, wherein
the nanoparticles comprise nanorods having a width between 5-15 nm and a length between 10-50 nm.

61. The method of claim 52, wherein
the phage is selected from the group consisting of M13, MS2, T4, T5, T7, K1F, K11, fd, f1 or SP6.

62. The method of claim 52, wherein
the receptor binding protein of the phage is derived from another phage type.

63. The method of claim 52, wherein
the target bacteria type is a gram negative bacteria and/or a drug-resistant bacteria.

64. A method of detecting bacterial cells of a selected type in a sample, comprising
applying a plurality of phages to a sample,
wherein each of the phages comprises
one or more receptor-binding proteins that confers specificity for the selected bacterial type,
and;
wherein a plurality of plasmonic nanoparticles have been conjugated to the phage or
wherein the phage is competent for functionalization with a selected plasmonic nanoparticle;
incubating the sample for a sufficient period of time for the functionalized phage to adsorb to bacterial cells of the selected type, if present in the sample;
if phages competent for functionalization with a selected plasmonic nanoparticle have been applied, performing the step of applying nanoparticles of the selected type to the sample under conditions that facilitate conjugation of the plasmonic nanoparticles to compatible moieties on the phage;
illuminating the sample with light energy sufficient to induce plasmon resonance excitation in the nanoparticles;
concurrently with the illumination step, measuring a selected optical signal wherein such optical signal is responsive to plasmon resonance excitation by nanoparticles aggregated by adsorption to bacterial cells of the selected type; and
by the use of an established relationship between optical signal value and the presence or abundance of bacterial cells of the selected type, the measured value of the optical signal is used to determine the presence or abundance of cells of the selected bacteria type in the sample.

65. The method of claim 64, wherein
the plasmonic nanoparticles are responsive to light of wavelengths between 650 and 2,500 nm.

66. The method of claim 64, wherein
the nanoparticles comprise a material selected from the group consisting of gold, silver, copper, aluminum, iron, iron oxides, zinc, cadmium, lanthanum, lead, tin, mercury, an alloy of the foregoing, functionalized carbon nanotubes or graphene, and a plasmonic organoometallic composition.

67. The method of claim 64, wherein
the nanoparticles comprise nanorods, nanostars, nanospheres, or nanoprisms.

68. The method of claim 67, wherein
the nanoparticles comprise nanorods, wherein the nanorods have an aspect ratio, measured as the length to width, of 2:1 to 10:1.

69. The method of claim 67, wherein
the nanoparticles comprise nanorods having a of width between 5-15 nm and length between 10-50 nm.

70. The method of claim 64, wherein
the one or more receptor binding proteins comprises a heterologous receptor binding protein derived from another phage type.

71. The method of claim 64, wherein
the optical signal is peak absorbance or color.

72. The method of claim 64, wherein,
the method comprises the additional step of isolating or concentrating bacterial cells from the sample following incubation, and, subsequently applying the illumination and measurement steps to a solution of isolated or concentrated bacterial cells in place of the sample.

73. The method of claim 23, wherein
the sample is selected from the group consisting of a clinical sample, an environmental sample, a food or agricultural sample, and cultured cells.


SEQUENCE LISTING (12 sequences)

SEQ ID NO: 1 - protein, 28 aa, Homo sapiens
KKKHRRRPSKKKRHWKPYYKLTWEEKKK

SEQ ID NO: 2 - protein, 9 aa, Human immunodeficiency virus 1
RKKRRQRRR

SEQ ID NO: 3 - protein, 33 aa, Homo sapiens
RIRAEMFAKGQPVAPYNTTQFLMDDHDQEEPDL

SEQ ID NO: 4 - protein, 48 aa, Human immunodeficiency virus 1
MEPVDPYLEPWKHPGSQPRTACNNCYCKKCCFHCYACFTRKGLGISYG

SEQ ID NO: 5 - protein, 5 aa, Artificial Sequence
  Description of Artificial Sequence Synthetic peptide
GGGGS

SEQ ID NO: 6 - protein, 121 aa, Artificial Sequence
  Description of Artificial Sequence Synthetic polypeptide
KKKHRRRPSKKKRHWKPYYKLTWEEKKKFDEKQSLRASRIRAEMFAKGQPVAPYNTTQFLMDDHDQEEPDLVDMEPVDPYLEPWKHPGSQPRTACNNCYCKKCCFHCYACFTRKGLGISYG

SEQ ID NO: 7 - protein, 111 aa, Artificial Sequence
  Description of Artificial Sequence Synthetic polypeptide
MEQKLISEEDLRKKRRQRRRAPPGGGGSRIRAEMFAKGQPVAPYNTTQFLMDDHDQEEPDLVDMEPVDPYLEPWKHPGSQPRTACNNCYCKKCCFHCYACFTRKGLGISYG

SEQ ID NO: 8 - protein, 93 aa, Artificial Sequence
  Description of Artificial Sequence Synthetic polypeptide
FDEKQSLRASRIRAEMFAKGQPVAPYNTTQFLMDDHDQEEPDLVDMEPVDPYLEPWKHPGSQPRTACNNCYCKKCCFHCYACFTRKGLGISYG

SEQ ID NO: 9 - protein, 78 aa, Artificial Sequence
  Description of Artificial Sequence Synthetic polypeptide
KKKHRRRPSKKKRHWKPYYKLTWEEKKKVDMEPVDPYLEPWKHPGSQPRTACNNCYCKKCCFHCYACFTRKGLGISYG

SEQ ID NO: 10 - DNA, 372 bp, Artificial Sequence
  Description of Artificial Sequence Synthetic polynucleotide
atggaattcaagaaaaaacataggagacgcccgtccaagaagaagcggcattggaaaccgtactacaagctgacctgggaagagaagaaaaagttcgacgagaaacagagccttcgagcttcaaggatccgagccgagatgttcgccaagggccagccggtcgcgccctataacaccacgcagttcctcatggatgatcacgaccaggaggagccggatctcgtcgacatggagccagtagatccatatctagagccctggaagcatccaggaagtcagcctaggactgcttgtaacaattgctattgtaaaaagtgttgctttcattgctacgcgtgtttcacaagaaaaggcttaggcatctcctatggc

SEQ ID NO: 11 - DNA, 279 bp, Artificial Sequence
  Description of Artificial Sequence Synthetic polynucleotide
ttcgacgagaaacagagccttcgagcttcaaggatccgagccgagatgttcgccaagggccagccggtcgcgccctataacaccacgcagttcctcatggatgatcacgaccaggaggagccggatctcgtcgacatggagccagtagatccatatctagagccctggaagcatccaggaagtcagcctaggactgcttgtaacaattgctattgtaaaaagtgttgctttcattgctacgcgtgtttcacaagaaaaggcttaggcatctcctatggc

SEQ ID NO: 12 - DNA, 303 bp, Artificial Sequence
  Description of Artificial Sequence Synthetic polynucleotide
aggaagaagcggagacagcgacgaagagctcctcctggtggaggaggctctaggatccgagccgagatgttcgccaagggccagccggtcgcgccctataacaccacgcagttcctcatggatgatcacgaccaggaggagccggatctcgtcgacatggagccagtagatccatatctagagccctggaagcatccaggaagtcagcctaggactgcttgtaacaattgctattgtaaaaagtgttgctttcattgctacgcgtgtttcacaagaaaaggcttaggcatctcctatggctag